(19) United States
(12) Patent Application Publication
(10) Pub. No.: US 2025/0340946 A1
(43) Pub. Date: Nov. 6, 2025

(54) METHOD OF PROGNOSING AND TREATING GLIOMA

(71) Applicants: Agency for Science, Technology and Research, Singapore (SG); Beijing Neurosurgical Institute, Beijing (CN)

(72) Inventors: Lele WU, Singapore (SG); Vinay TERGAONKAR, Singapore (SG); Bilal ÜNAL, Singapore (SG); Tao JIANG, Beijing (CN); Zhang ZHAO, Beijing (CN)

(73) Assignees: Agency for Science, Technology and Research, Singapore (SG); Beijing Neurosurgical Institute, Beijing (CN)

(21) Appl. No.: 18/860,293

(22) PCT Filed: Mar. 23, 2023
(86) PCT No.: PCT/SG2023/050192

§ 371 Date: Oct. 25, 2024

(30) Foreign Application Priority Data
Apr. 26, 2022 (SG) 10202204369Y

(51) Int. Cl. C12Q 1/6886; A61K 31/495; A61K 45/06

(52) U.S. Cl. CPC C12Q 1/6886; A61K 31/495; A61K 45/06; C12Q 2600/106; C12Q 2600/118; C12Q 2600/158

(57) ABSTRACT

The present invention relates to a method comprising the detection of LOC105375914 RNA in a glioma sample obtained from a subject, wherein an elevated level of LOC105375914 RNA as compared to a reference indicates that the subject has a high grade glioma, has a likelihood of resistance to chemotherapy, has a likelihood of recurrence, and/or is likely to have a poor prognosis. The present invention also relates to a method of treating a glioma expressing LOC105375914 RNA in a subject by administering an inhibitor of the LOC-DEAH-box helicase 15 (DHX15) complex to the subject.

### FIELD

The present invention relates generally to the field cancer detection and treatment. In particular, the specification teaches methods of prognosing, identifying and treating glioma in a subject.

### BACKGROUND

Cancer is the second leading cause of death in the United States after cardiovascular disease. Gliomas are brain tumors that start in glial cells, which are supporting cells of the brain and the spinal cord. Glial cells include astrocytes, oligo dendrocytes and ependymal cells. Astrocytomas are tumors that affect astrocytes and are the most common type of glioma in both adults and children. The most widely used scheme for classification and grading of gliomas is that of the World Health Organization where they are classified according to their degree of malignancies on a scale of I to IV. Astrocytomas can be low grade (i.e. grade I or II) or high grade (grade III or IV). Grade 4 astrocytomas are also called glioblastoma or glioblastoma multiforme (GBM).

Isocitrate dehydrogenases (IDHs), such as IDH1, are frequently mutated in a broad spectrum of cancers, including gliomas. Surprisingly, glioma patients harbouring WT-IDH1 (wild-type IDH1) exhibit worse overall survival than patients with IDH1 mutations. Hence, there is still in an urgent need to identify novel therapeutic strategies especially for high-grade WT-IDH1 gliomas. Thus, understanding the molecular players/signalling which get activated and lead to worse outcomes in WT-IDH1 gliomas may help to design effective targeting strategies specifically for WT-IDH1 gliomas.

A major pathway activated in these cancers is the NFκB signalling pathway. However, to date, no NFκB inhibitors have been clinically approved because blocking this pathway leads to massive toxicity due to the involvement of NFκB in many housekeeping functions. Thus, identifying “context specific” regulators of NFκB signalling which may led to new therapeutic targets.

It would be desirable to overcome or ameliorate at least one of the above-described problems, or at least to provide a useful alternative.

### SUMMARY

Disclosed herein is a method of determining the prognosis of a glioma in a subject, the method comprising detecting LOC105375914 RNA in a glioma sample obtained from the subject, wherein an elevated level of LOC105375914 RNA as compared to a reference indicates that the subject has a high grade glioma and/or is likely to have a poor prognosis.

Disclosed herein is a method of identifying a high grade glioma in a subject, the method comprising: detecting LOC105375914 RNA in a cancer sample obtained from the subject, wherein an elevated level of LOC105375914 RNA as compared to a reference indicates that the subject has a high grade glioma.

Disclosed herein is a method of identifying and treating a high grade glioma in a subject, the method comprising: a) detecting LOC105375914 RNA in a cancer sample obtained from the subject, wherein an elevated level of LOC105375914 RNA as compared to a reference indicates that the subject has a high grade glioma; and b) administering an anti-cancer agent to the subject found to have high grade glioma to treat the high grade glioma.

Disclosed herein is a method of predicting a likelihood of resistance to chemotherapy in a subject suffering from a glioma, the method comprising detecting LOC105375914 RNA in a glioma sample obtained from the subject, wherein an elevated level of LOC105375914 RNA as compared to a reference indicates that the subject has a likelihood of resistance to chemotherapy.

Disclosed herein is a method of predicting a likelihood of recurrence of a glioma in a subject, the method comprising detecting LOC105375914 RNA in a glioma sample obtained from the subject, wherein an elevated level of LOC105375914 RNA as compared to a reference indicates that the subject has a likelihood of recurrence.

Disclosed herein is a method of treating a glioma in a subject by administering an inhibitor of the LOC-DEAH-box helicase 15 (DHX15) complex to the subject.

Disclosed herein is a method of inhibiting proliferation of a glioma stem cell in a subject, the method comprising administering an inhibitor of the LOC-DEAH-box helicase 15 (DHX15) complex to the subject.

### BRIEF DESCRIPTION OF DRAWINGS

Embodiments of the present invention are hereafter described, by way of non-limiting example only, with reference to the accompanying drawings in which:

FIG. 1: LOC was identified as a novel NFκB regulator by high-throughput lncRNA siRNA screening. A) Over-represented KEGG pathways of genes that are significantly differentially expressed in wtIDH1 glioma patients are compared to mIDH glioma patients. B) Averaged relative luminescence of lncRNA siRNA screening are shown. LOC and positive control RIPK1 are highlighted. C) LOC expression in normal brain tissues and different types of gliomas based on RNA-seq data from CGGA. Abbreviation: O: Oligodendro, rO: recurrent Oligodendro, OA: Oligodendro and Astrocytoma, rOA: recurrent Oligodendro and Astrocytoma, A: Astrocytoma, rA: recurrent Astrocytoma, AO: Anaplastic Oligodendro, rAO: recurrent Anaplastic Oligodendro, AOA: Anaplastic Oligodendro and Astrocytoma, rAOA: recurrent Anaplastic Oligodendro and Astrocytoma, AA: Anaplastic Astrocytoma, rAA: recurrent Anaplastic Astrocytoma, GBM: glioblastoma, rGBM: recurrent glioblastoma, sGBM: second glioblastoma, Control: Healthy control. D) Kaplan Meier survival curve of GBM patients from the LOC-high (n=108) or LOC-low group (n=113). E) RT-qPCR analysis of LOC expression in GBM patients with high LOC expression (LOC-high) (n=20) or low LOC expression (LOC low) (n=20). Top and bottom 20 patients were selected from a set of 59 GBM patients. Data was normalized to GAPDH and the normalized AACt values are shown in log 10 scale. P-Value was calculated by student t-test method. F) Kaplan Meier survival curve of GBM patients from the LOC-high or LOC-low group. p-value was calculated using Gehan-Breslow-Wilcoxon test. G-H) MRI images from cranium of 3 independent GBM patients in each group before operation day (POD), on day of surgery (OP) and after surgical dissection followed by TMZ (TMZ) and CCRT (chemo-radiation therapy) treatment. Treatment histories and tumor phylogenies of G) patients with LOC-high expression and H) patients with LOC-low expression. Circles on the bar represent days on which the presented MRI scans were obtained. The circles on the images indicate the areas where tumor tissue was resected. Abbreviations: POD, previous-operation day; OP, operation process. The number of days has been indicated as −1D to +245D.

FIG. 2: LOC promotes GBM tumorigenesis in vitro and in vivo. A) RT-qPCR analysis of LOC expression in GBM patient-derived cells with LOC knockdown by lentivirus delivery. B) GBM patient-derived primary cells treated with DMSO or TMZ together with Control shRNA or two independent shRNA targeting LOC. Cell viability was measured by ATPlite assay and data was normalized to DMSO treated control shRNA transduced cells. C) Limiting dilution assay (LDA) for in vitro tumorsphere formation in GBM patient-derived cells with LOC knockdown. LDA clonogenic significance is measured by the linear regression analysis. D) Patient-derived GBM cells were infected with control shRNA, LOC shRNA #1 and LOC shRNA #2 vectors. Cells with or without LOC knockdown were intracranially injected to mice (n=8) and analyzed for survival. E) Representative H&E sections of the mouse brains from D. Bar, 2 mm. F) Immunofluorescence staining of cancer stem cell marker Nestin in PDX model derived tumor samples. G) Quantification of immunofluorescence staining of cancer stem cell marker Nestin.

FIG. 3: LOC correlates with infiltration of GAMs in GBM tumor ecosystem. A) Flow chart of next generation sequencing including whole-exome sequencing, scRNA-seq and bulk RNA-seq from matched GBM patients. B) Mutational profile of hotspot mutation in eight GBM patient samples. C) Uniform manifold approximation and projection (UMAP) representation of all cell populations detected in glioblastoma patients and a patient with lung squamous cell carcinoma. lung squamous cell carcinoma samples used as a control. D) Bar plot displaying the relative proportions of each cell type in six glioblastomas patients which can be separated into groups of LOC-low (S5, S13, S3) and LOC-high (S2, S4, S7). GAMs, Glioblastoma-associated macrophages and microglia. E) Box plot depicts the comparison of relative cell type proportion between patients with low and high LOC expression. Student t test's p-value (5.38×10e−4) indicate significant difference in cell proportion of GAMs between the two groups of patients. F) UMAP representation of all cell populations detected in glioblastoma patient, this dataset is from 10× genomics website. G) LOC expression in different cell populations obtained from F).

FIG. 4: Deletion of Gm16685 from both tumor and host compartments leads to most profound frequency of tumor regressions. A) Schematic representation of syngeneic model. Gm16685 WT or KO GL261-Luc cells were intracranially injected into Gm16685 WT and KO mice (WT-WT stands for WT GL261-Luc cells were injected into WT mice, WT-KO stands for WT GL261-Luc cells were injected into KO mice, KO-WT stands for KO GL261-Luc cells were injected into WT mice, KO-KO stands for KO GL261-Luc cells were injected into KO mice. After 3 weeks, tumor formation was monitored by bioluminescence imaging. B-C) Bioluminescence imaging of tumor size from each group and B) Kaplan-Meier survival analysis C) of syngeneic model with indicated conditions (n=6 or 8 mice per group). p-value was calculated using Gehan-Breslow-Wilcoxon test. D-E) Immunofluorescence staining of glioma cells marker GFAP D) in syngeneic model derived tumor samples. E) Quantification of immunofluorescence staining of glioma cells marker GFAP. F-G) Immunofluorescence staining of GAMs marker IBA1 F) in syngeneic model derived tumor samples. G) Quantification of immunofluorescence staining of GAMs marker IBA1.

FIG. 5: Helicase activity of DHX15 is essential for LOC mediated squelching of PPM1 away from NFκB p65 subunit. A) DHX15 expression in normal brain tissues and different WHO grade gliomas based on RNA-seq data from CGGA. B) Kaplan Meier survival curve of GBM patients from DHX15-high or DHX15-low group. C) 293T WT and LOC KO cells were transfected with control vector (Ctrl vector) or Flag-DHX15 as indicated. DHX15 was immunoprecipitated by flag antibody and co-purified proteins were analyzed by western blotting using PPM1D and flag antibodies. D) LN18 WT and LOC KO cells were treated with TNFα for the indicated time points and endogenous DHX15 or p65 was immunoprecipitated with antibody against DHX15 or p65. IP Samples were analyzed by subsequent immunoblot for the indicated proteins. E) Flag-tagged WT (Flag-WT-DHX15) or helicase dead mutant (Flag-Mut-DHX15) DHX15 or control vector (Ctrl Vector) were ectopically expressed in 293T cells and stimulated with TNFα for the indicated time points. DHX15 was immunoprecipitated by flag antibody and co-purified proteins were analyzed by western blotting. F) LN18 WT cells were treated with TNFα for the indicated time points and endogenous DHX15 or DKC were immunoprecipitated with antibody against DHX15 or DKC. DHX15 or DKC RIP followed by RT-qPCR shows the enrichment of co-eluted LOC. DKC acts as a negative control. Graph shows the fold enrichment which was normalized to IgG. G) Graph shows TNFα gene expression in LN18 WT and LOC KO cells transfected with empty vector (Ctrl Vector) or Flag-tagged-WT-DHX15 (WT-DHX15) or Flag-tagged-mut-DHX15 (mut-DHX15) and stimulated with TNFα for 90 min. Data was normalized to actin. **, p<0.01; ***, p 973<0.001. p-values were calculated by two-tailed student's t-test method. H) WT and LOC KO LN18 cells were transfected with Control siRNA or PPM1D siRNA. After 72 h post-transfection, cells were treated with or without TNFα and harvested for western blot. cell lysates were analysed via western blot for the indicated proteins.

FIG. 6: LOC: DHX15 serves as a targetable vulnerability in wtIDH1 high-grade glioma. A) LOC expression in different IDH mutation status across different WHO grade gliomas based on RNA-seq data from CGGA. B) qPCR of LOC expression in wtIDH1 LN18 cells or mIDH1 clones. Those clones were generated by base editing. C) qPCR of LOC expression in wtIDH1 and mIDH1 LN18 cells treated with or without mIDH1 inhibitor AGI-5198. D) qPCR of LOC expression in wtIDH1 and mIDH1 LN18 cells with or without DMNT inhibitor 5-Azac treatment. E) Protein lysates from wtIDH1 and mIDH1 LN18 cells were analyzed for levels of phosphorylated and total p65 and p38 proteins by western blot. F) wtIDH1 and mIDH1 LN18 cells were transfected with empty vector (EV) or expression vectors of LOC or mutant version of LOC (LOC-M2). After 48 hours cells were harvested and endogenous DHX15 or p65 was immunoprecipitated with antibody against DHX15 or p65. IP samples were analyzed by subsequent immunoblot for the indicated proteins at indicated time points. G-H) Cell viability assay of wtIDH1 G) and mIDH1 LN18 cells H) treated with or without DHX inhibitor. I) Fluorescence imaging of wtIDH1 and mIDH1 GBM orthotopic xenograft models with or without DHX inhibitor treatment. J) Quantification of tumor signal intensity obtained from I). K-J) Kaplan-Meier survival analysis of wtIDH1 GBM xenograft model K) or mIDH1 GBM xenograft model L) with or without DHX inhibitor treatment.

FIG. 7: LOC: DHX15-PPMID-NFκB axis confers TMZ resistance. A-B) WT LN18 cells were transfected with Ctrl siRNA or LOC siRNA A) or DHX15 siRNA B). After 72 h post transfection, cells were treated with or without TMZ. Cell viability was analyzed by CCK8 kit. C) WT U251 cells were transfected with Ctrl Vector or expression vector of LOC. After 48 h post-transfection, cells were treated with or without TMZ. Cell viability was analyzed by CCK8 kit. D) WT LN18 cells were treated with TMZ or DHX inhibitor or combination treatment. Cell viability was analyzed by CCK8 kit. E) Fluorescence imaging of wtIDH1 GBM orthotopic xenograft models treated with DHX inhibitor, TMZ, or combination treatment. F) Quantification of tumor signal intensity obtained from E). G) Kaplan-Meier survival analysis of wtIDH1 GBM xenograft model treated with TMZ or DHX inhibitor or combination treatment. H) RT-qPCR analysis of MGMT expression in GBM patient-derived cells with LOC knockdown by lentivirus delivery. I) RT-qPCR analysis of MGMT expression in LN18 cells with LOC knockdown by siRNA. J) RT-qPCR analysis of MGMT expression in GBM patient-derived cells with LOC knockdown or overexpression of LOC expression in knockdown group. K) RT-qPCR analysis of MGMT expression in WT LN18 cells with or without DHX inhibitor treatment. L) WT LN18 cells were transfected with Ctrl Vector or expression vector of MGMT. After 48 h post-transfection, cells were treated with or without TMZ and DHX inhibitor combination treatment. Cell viability was analyzed by CCK8 kit.

FIG. 8 shows A) LOC expression is lost in mutant IDH1 gliomas due to mutant IDH1 mediated hypermethylation phenotype. B) in WT-IDH1 high-grade glioma, LOC can be unfolded by RNA helicase DHX15 to activate NFκB signalling which leads to tumor growth and chemo-resistance of WT-IDH1 gliomas. C) Inhibition of LOC unfolding by DHX-inhibitor will blunt NFκB signalling which can dampen tumor growth and chemo-resistance of WT-IDH1 gliomas.

FIG. 9 shows the combination therapy of TMZ with another 3 RNA helicase inhibitors: Rocaglamide, DDX3-IN and RK-33. Rocaglamide, DDX3-IN and RK-33 shows inhibition of cell viability in GBM cells. However, combination therapy of TMZ with Rocaglamide, DDX3-IN and RK-33 does not show synergistic inhibition of cell viability.

FIG. 10: wtIDH1 glioma exhibits higher NFκB activity. A) Mutation landscape of hotspot mutation in glioma patients based on whole exome sequencing data from 1018 CGGA. B) Kaplan Meier survival curve of wtIDH1 and mIDH1 glioma patients based on clinical data from CGGA. C) Volcano plot of TNF/NFκB signalling genes that are significantly differentially expressed in GBM compared to healthy control.

FIG. 11: LOC was highly expressed in high-grade glioma. A) LOC expression in normal brain tissues and different WHO grade gliomas based on RNA-seq data from CGGA. B) LOC expression different WHO grade gliomas based on RNA-seq data from TCGA. C) RT-qPCR of LOC expression in 59 GBM patients. ddCt was normalized by Gapdh.

FIG. 12: LOC regulates NFκB/p38 activation and target gene expression. A) Schematic view of LOC lncRNA promoter targeting with CRISPR-Cas9 editing (inside IL7-intron). NFκB binding motifs were shown as yellow in the promoter region of LOC. B-C) Expression analysis was performed by RT-qPCR for B) TNFα and C) LOC in T98G treated with DMSO, IKK2 inhibitor in the presence or absence of TNFα stimulation. D-F) LN18 wild type (WT) and LOC KO (KO) cells were stimulated with TNFα for the indicated duration. Gene expression was analyzed by RT-qPCR for D) LOC and E) TNFα. F) Protein lysates were analyzed for levels of phosphorylated and total p65 and p38 proteins by western blot. G-I) LN18 cells were transfected with control or LOC siRNA and after 72 h, stimulated with TNFα for indicated duration and RNA was analyzed by RT-qPCR for G) LOC transcript and H) TNFα transcripts. I) Total cell lysates were analyzed by western blot with the indicated antibodies. L) T98G WT and LOC KO (KO) cells were stimulated with TNFα for indicated time points. Gene expression was analyzed by RT-qPCR for J) LOC and K) TNFα. L) Cell lysates were analyzed by western blot for the indicated proteins.

FIG. 13: LOC: DHX15 axis is required for NFκB activation. A) T98G WT and LOC KO cells were treated with TNFα for the indicated time points and endogenous DHX15 or p65 was immunoprecipitated with antibody against DHX15 or p65. IP samples were analyzed by subsequent immunoblot for the indicated proteins. B) Graph shows TNFα gene expression in T98G WT and LOC KO cells transfected with empty vector (Ctrl Vector) or Flag tagged-WT-DHX15 (WT-DHX15) or Flag-tagged-mut-DHX15 (mut-DHX15) and stimulated with TNFα for 90 min. Data was normalized to actin. **, p<0.01; ***, p<0.001; n.s., not significant. p-values were calculated by two-tailed student's t-test method.

FIG. 14: LOC expression is dampened in mIDH1 gliomas. A) LOC expression in gliomas with or without IDH1 mutation based on the RNA-seq data from TCGA.B) Sanger sequencing results of wtIDH1 and mIDH1 clones created by base editing. C) Protein lysates were analyzed for IDH1 (R132H) by western blot using anti-IDH1 (R132H). D) wtIDH1 and mIDH1 LN18 cells were treated with or without DHX inhibitor. DHX15 or p65 was immunoprecipitated with antibody against DHX15 or p65. IP samples were analyzed by subsequent immunoblot for the indicated proteins.

FIG. 15: LOC: DHX15 contributes to TMZ resistance. A-B) WT T98G cells were transfected with Ctrl siRNA or LOC siRNA A) or DHX15 siRNA B). After 72 h post-transfection, cells were treated with or without TMZ. Cell viability were analyzed by CCK8 kit. C) WT SF295 cells were transfected with Ctrl Vector or expression vector of LOC. After 48 h post-transfection, cells were treated with or without TMZ. Cell viability were analyzed by CCK8 kit. D) WT T98G cells were treated with TMZ or DHX inhibitor or combination treatment. Cell viability were analyzed by CCK8 kit.

### DETAILED DESCRIPTION

The present specification teaches a method of determining the prognosis of a cancers in a subject. Provided herein are methods and compositions using non-coding RNAs for determining the prognosis of a cancer in a subject. In particular, there is providing of using non-coding RNAs for determining the prognosis of a glioma in a subject.

Disclosed herein is a method of determining the prognosis of a glioma in a subject, the method comprising detecting LOC105375914 RNA in a glioma sample obtained from the subject, wherein an elevated level of LOC105375914 RNA as compared to a reference indicates that the subject has a high grade glioma and/or is likely to have a poor prognosis.

Without being bound by theory, the inventors have performed a high throughput screen of long non-coding RNAs (lncRNAs) to identify novel regulators of NFκB from the non-coding genome. The inventors have found that RNA LOC105375914 (LOC) is a novel component of NFκB signaling. LOC expression is regulated by WT-IDH1 and is lost in mutant IDH1 gliomas. Once activated by IDH1, LOC positively regulates NFκB activation and glioma progression. It was identified that for LOC to function as an activator of NFκB and promote gliomagenesis, it requires to be unfolded by the action of a specific ATP dependent RNA helicase, DHX15. Unwinding of LOC by DHX15 RNA helicase is required for NFκB activity and growth and chemo-resistance of WT-IDH1 gliomas. Targeting LOC-DHX15 complex by blocking RNA helicase activity of DHX15 using small molecule inhibitors exerts synergistic effect with temozolomide (TMZ), the current standard of care for gliomas. The term “prognosis” as referred to herein refers to a prediction of the probable course and outcome of a clinical condition or disease. A prognosis of a patient is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis may be expressed as the number of months, on average, that a patient can expect to survive as a result of a condition or disease. The prognosis of a patient may be considered as an expression of relativism, with many factors effecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

The term “tumor,” as used herein, refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, glioma, breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer, carcinoma, melanoma, brain cancer, non-small cell lung cancer, squamous cell cancer of the head and neck, endometrial cancer, multiple myeloma, rectal cancer, and esophageal cancer. In one embodiment, the cancer is a glioma. In one embodiment, the cancer is a metastatic cancer. In one embodiment, the cancer is a chemo-resistant cancer. The chemo-resistant cancer may, for example, be a TMZ resistant glioma.

The term “glioma” is used herein in accordance with its normal usage in the art and refers to a tumor that arises from glial cells or their precursors of the brain or spinal cord. Glioma includes a variety of different tumor types, including, but not limited to gliomas, glioblastoma multiforme (GBM), astrocytomas, and oligodendrogliomas.

In one embodiment, the high grade glioma is a WT-IDH1 glioma. In one embodiment, the high grade glioma is a World Health Organization (WHO) Grade III or IV glioma. In one embodiment, the high grade glioma is Glioblastoma multiforme (GBM).

The terms “subject,” “individual,” and “patient”-which are used interchangeably herein, are intended to refer to any subject, preferably a mammalian subject, and more preferably still a human subject, for whom therapy or prophylaxis desired. Mammalian subjects include humans, domestic animals, farm animals, sports animals, and zoo animals including, e.g., humans, non-human primates, dogs, cats, mice, rats, guinea pigs, and the like. In most of the embodiments, the subject has, or is suspected of having, a glioma, such as glioblastoma multiforme (GBM), an astrocytoma, or an oligodendroglioma.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present disclosure.

A sample can be a biological sample which refers to the fact that it is derived or obtained from a living organism. The organism can be in vivo (e.g. a whole organism) or can be in vitro (e.g., cells or organs grown in culture). A “biological sample” also refers to a cell or population of cells or a quantity of tissue or fluid from a subject. Most often, a sample has been removed from a subject, but the term “biological sample” can also refer to cells or tissue analyzed in vivo, i.e., without removal from the subject. Often, a “biological sample” will contain cells from a subject, but the term can also refer to non-cellular biological material, such as non-cellular fractions of blood, saliva, or urine. The biological sample may be from a resection, bronchoscopic biopsy, or core needle biopsy of a primary, secondary or metastatic tumor, or a cellblock from pleural fluid. In addition, fine needle aspirate biological samples are also useful. In one embodiment, a biological sample is ascites. Biological samples also include explants and primary and/or transformed cell cultures derived from patient tissues. A biological sample can be provided by removing a sample of cells from subject, but can also be accomplished by using previously isolated cells or cellular extracts (e.g. isolated by another person, at another time, and/or for another purpose). Archival tissues, such as those having treatment or outcome history may also be used. Biological samples include, but are not limited to, tissue biopsies, scrapes (e.g. buccal scrapes), whole blood, plasma, serum, urine, saliva, cell culture, or cerebrospinal fluid.

The term “reference” may refer to a sample from a healthy individual (such as one who does not have a glioma) or may refer to a non-cancerous sample. It may also refer to a pre-determined value.

As used herein, the term “elevated or “increased” with reference to the level of LOC105375914 RNA refers to a statistically significant and measurable increase in the level of LOC105375914 RNA as compared to a reference. The increase is preferably an increase of at least about 10%, or an increase of at least about 20%, or an increase of at least about 30%, or an increase of at least about 40%, or an increase of at least about 50%.

In one embodiment, an elevated or increased level of LOC105375914 RNA as compared to a reference indicates that the subject has a high grade glioma and/or is likely to have a poor prognosis. The increase in level may be an increase of 1.1 times, 1.2 times, 1.3 times, 1.4 times, 1.5 times, 1.6 times, 1.7 times, 1.8 times, 1.9 times, 2 times, 3 times, 4 times, 5 times, 6 times, 7 times, 8 times, 9 times, 10 times, 11 times 12 times, 13 times, 14 times, 15 times, 16 times, 17 times, 18 times, 19 times, 20 times, 21 times, 22 times, 23 fold, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times or 100 times or anywhere in between as compared to a reference.

Any patient sample suspected of containing a non-coding RNA as defined herein may be tested according to methods of the present disclosure. By way of non-limiting examples, the sample may be tissue (e.g., a biopsy sample), blood, plasma, serum, urine, saliva, cell culture or cerebrospinal fluid.

In some embodiments, the patient sample is subjected to preliminary processing designed to isolate or enrich the sample for the non-coding RNA or cells that contain the non-coding RNA. A variety of techniques known to those of ordinary skill in the art may be used for this purpose, including but not limited to: centrifugation; immunocapture; cell lysis; nucleic acid amplification; and, nucleic acid target capture. The non-coding RNAs may be detected along with other markers in a multiplex or panel format.

Markers may be selected for their predictive value alone or in combination with non-coding RNA described herein. Markers for other cancers, diseases, infections, and metabolic conditions are also contemplated for inclusion in a multiplex or panel format.

As used herein, the terms “detect”, “detecting” or “detection” may describe either the general act of discovering or discerning or the specific observation of a composition. Detecting a composition may comprise determining the presence or absence of a composition. Detecting may comprise quantifying a composition. For example, detecting comprises determining the expression level of a composition. The composition may comprise a nucleic acid molecule. For example, the composition may comprise at least a portion of the ncRNAs disclosed herein. Alternatively, or additionally, the composition may be a detectably labeled composition.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “polynucleotide” or “nucleic acid” are used interchangeably herein to refer to a polymer of nucleotides, which can be mRNA, RNA, CRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

As used herein, the term “oligonucleotide,” refers to a short length of single-stranded polynucleotide chain. Oligonucleotides are typically less than 200 residues long (e.g. between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

The term “label” as used herein refers to any atom or molecule that can be used to provide a detectable (preferably quantifiable) effect, and that can be attached to a nucleic acid or protein. Labels include but are not limited to dyes; radiolabels such as 2P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable. In some embodiments, nucleic acids are detected directly without a label (e.g., directly reading a sequence).

As used herein, the terms “complementary” or “complementarity” are used in reference to polynucleotides (i.e., a sequence of nucleotides) related by the base-pairing rules. Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that nonspecific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G: C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

The non-coding RNA of the present disclosure may be detected using a variety of nucleic acid techniques known to those of ordinary skill in the art, including but not limited to: nucleic acid sequencing; nucleic acid hybridization; and, nucleic acid amplification.

In some embodiments, nucleic acid sequencing methods are utilized (e.g., for detection of amplified nucleic acids). In some embodiments, the technology provided herein finds use in a Second Generation (i.e. Next Generation or Next-Gen), Third Generation (i.e. Next-Next-Gen), or Fourth Generation (i.e. N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology. Those of ordinary skill in the art will recognize that because RNA is less stable in the cell and more prone to nuclease attack experimentally RNA is usually reverse transcribed to DNA before sequencing.

Provided herein are also means for correlating the level of non-coding RNAs being studied with a prognosis of disease outcome. Such means may comprise one or more of a variety of correlative techniques, including lookup tables, algorithms, multivariate models, and linear or nonlinear combinations of expression models or algorithms. The levels may be converted to one or more likelihood scores, reflecting a likelihood that the patient providing the sample may exhibit a particular disease outcome. The models and/or algorithms can be provided in machine readable format and can optionally further designate a treatment modality for a patient or class of patients.

Also provided herein are output means for outputting the disease status, prognosis and/or a treatment modality. Such output means can take any form which transmits the results to a patient and/or a healthcare provider, and may include a monitor, a printed format, or both. A computer system may be used for performing one or more of the steps provided.

The method as defined herein may comprise detecting wild-type isocitrate dehydrogenase 1 (IDH1). Methods for detecting wild-type or mutant IDH1 nucleic acid or polypeptides are well known in the art. In one embodiment, the method as defined herein comprises detecting an elevated level of LOC105375914 RNA and wild-type isocitrate dehydrogenase 1 (IDH1).

The method as defined herein may comprise detecting NFKB Inhibitor Alpha (NFKBIA). The method as defined herein may comprise detecting a deletion in the NFKBIA gene or a decreased level of NFKBIA expression.

The terms “protein” and “polypeptide” are used interchangeably and refer to any polymer of amino acids (dipeptide or greater) linked through peptide bonds or modified peptide bonds. Polypeptides of less than about 10-20 amino acid residues are commonly referred to as “peptides.” The polypeptides of the invention may comprise non-peptidic components, such as carbohydrate groups. Carbohydrates and other non-peptidic substituents may be added to a polypeptide by the cell in which the polypeptide is produced, and will vary with the type of cell. Polypeptides are defined herein, in terms of their amino acid backbone structures; substituents such as carbohydrate groups are generally not specified, but may be present nonetheless.

In one embodiment, the high grade glioma is likely to be resistant to chemotherapy.

In one embodiment, the high grade glioma has a likelihood of cancer recurrence following cancer therapy.

Disclosed herein is a method of identifying a high grade glioma in a subject, the method comprising: a) detecting LOC105375914 RNA in a cancer sample obtained from the subject, wherein an elevated level of LOC105375914 RNA as compared to a reference indicates that the subject has a high grade glioma.

In one embodiment, the method stratifies a subject as one having a high grade glioma or a low grade glioma.

Disclosed herein is a method of predicting a likelihood of resistance to chemotherapy in a subject suffering from a glioma, the method comprising detecting LOC105375914 RNA in a glioma sample obtained from the subject, wherein an elevated level of LOC105375914 RNA as compared to a reference indicates that the subject has a likelihood of resistance to chemotherapy.

Disclosed herein is a method of predicting a likelihood of recurrence of a glioma in a subject, the method comprising detecting LOC105375914 RNA in a glioma sample obtained from the subject, wherein an elevated level of LOC105375914 RNA as compared to a reference indicates that the subject has a likelihood of recurrence.

The term “recurrence” as used herein may refer to a cancer that has recurred (come back), usually after a period of time during which the cancer could not be detected. The cancer may be called a recurrent cancer. The recurrent cancer may come back to the same place as the original (primary) tumor or to another place in the body. The recurrence may be considered a “local recurrence” when the cancer is in the same place as the original cancer or very close to it. The recurrence may be a “regional recurrence” when the tumor has grown into lymph nodes or tissues near the original cancer. The recurrence may be called a distant recurrence when the cancer has spread to organs or tissues far from the original cancer. When the cancer spreads to a distant place in the body, the recurrent cancer may be called metastasis or metastatic cancer.

The term “likelihood of recurrence” may refer to how likely it is for a cancer to recur in a subject. An increased level of LOC105375914 RNA as compared to a reference may indicate a 10%, 20%, 30%, 40%, 50%, 60%, 70%, 80%, 90% or 99% or more likelihood recurrence in the subject.

Disclosed herein is a method of identifying and treating a high grade glioma in a subject, the method comprising: a) detecting LOC105375914 RNA in a cancer sample obtained from the subject, wherein an elevated level of LOC105375914 RNA as compared to a reference indicates that the subject has a high grade glioma; and b) administering an anti-cancer agent to the subject found to have high grade glioma to treat the high grade glioma.

In one embodiment, the method comprises treating a subject. The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

In carrying out the treatment and/or prevention methods described herein, any suitable method or route of administration can be used to deliver the active agents. In some embodiments systemic administration may be employed. “Systemic administration” means that the active agent is administered such that it enters the circulatory system, for example, via enteral, parenteral, inhalational, or transdermal routes. Enteral routes of administration involve the gastrointestinal tract and include, without limitation, oral, sublingual, buccal, and rectal delivery. Parenteral routes of administration involve routes other than the gastrointestinal tract and include, without limitation, intravenous, intramuscular, intraperitoneal, intrathecal, and subcutaneous.

In some embodiments (including, but not limited to, those in which one or more of the agents used is not able to permeate the blood-brain barrier), local administration may be employed. “Local administration” means that a pharmaceutical composition is administered directly to where its action is desired (e.g., at or near the site of a glioma), for example via intracranial (e.g. intracerebral) delivery, such as via direct intratumoral injection. For example, in some embodiments pressure-driven infusion through an intracranial catheter, also known as convection-enhanced delivery (CED) may be used.

It is within the skill of one of ordinary skill in the art to select an appropriate route of administration taking into account the nature of the specific active agent being used and nature of the specific glioma to be treated.

As used herein the terms “effective amount” or “therapeutically effective amount” refer to an amount of an active agent as described herein that is sufficient to achieve, or contribute towards achieving, one or more desirable clinical outcomes, such as those described in the “treatment” and “prevention” descriptions above. An appropriate “effective” amount in any individual case may be determined using standard techniques known in the art, such as dose escalation studies, and may be determined taking into account such factors as the desired route of administration (e.g. systemic vs. intracranial), desired frequency of dosing, etc. Furthermore, an “effective amount” may be determined in the context of the co-administration method to be used.

The method may comprise administering an effective amount of an anti-cancer agent to the subject. The anti-cancer agent may be a standard-of-care chemotherapy. The anti-cancer agent may be temozolomide (TMZ).

As used herein, Temozolomide (TMZ),” also known as Temodar® and Temodal®, is an oral alkylating agent. TMZ is a derivative of imidazotetrazine, and is the prodrug of MTIC (3-methyl-(triazen-1-yl) imidazole-4-carboxamide). TMZ undergoes rapid chemical conversion in the systemic circulation at physiological pH to the active compound, MTIC (monomethyl triazeno imidazole carboxamide). A non-limiting example of a TMZ analog is MTIC. Other examples of TMZ analogs are disclosed in, e.g., U.S. Pat. Nos. 6,844,434 and 7,087,751.

In one embodiment, the anti-cancer agent is a chemotherapeutic agent. The anti-cancer agent may be an alkylating agent. Exemplary alkylating agents include, but are not limited to, mechlorethamine, cyclophosphamide, ifosamide, melphalan, chlorambucil, busulfan, and thiotepa as well as nitrosurea alkylating agents such as carmustine and lomustine. In one embodiment, the anti-cancer agent is a platinum drug. Exemplary platinum drugs include, but are not limited to, cisplatin, carboplatin, oxaliplatin, satraplatin, picoplatin, nedaplatin, triplatin, and lipoplatin. In one embodiment, the anti-cancer agent is an antimetabolite. Exemplary antimetabolites include, but are not limited to, 5-fluorouracil (5-FU), 6-mercaptopurine (6-MP), capecitabine (Xeloda®), cytarabine (Ara-C®), floxuridine, fludarabine, gemcitabine (Gemzar®), hydroxyurea, methotrexate, and pemetrexed (Alimta®). In one embodiment, the anti-cancer agent is an anti-tumor antibiotic. Anthracyclines are anti-tumor antibiotics that interfere with enzymes involved in DNA replication. Exemplary anthracyclines include, but are not limited to, daunorubicin, doxorubicin, epirubicin, and idarubicin. Other anti-tumor antibiotics include actinomycin-D, bleomycin, mitomycin-C, and mitoxantrone. In one embodiment, the anti-cancer agent is a topoisomerase inhibitor. Exemplary toposiomerase inhibitors include, but are not limited to, doxorubicin, topotecan, irinotecan (CPT-11), etoposide (VP-16), teniposide, and mitoxantrone. In one embodiment, the anti-cancer agent is a mitotic inhibitor. Exemplary mitotic inhibitors include, but are not limited to, paclitaxel (Taxol®), docetaxel (Taxotere®), ixabepilone (Ixempra®), vinblastine (Velban®), vincristine (Oncovin®), vinorelbine (Navelbine®), and estramustine (Emcyt®). In one embodiment, the anti-cancer agent is a platinum-based chemotherapeutic agent, such as oxaliplatin.

Disclosed herein is a method of treating a glioma in a subject by administering an inhibitor of the LOC-DEAH-box helicase 15 (DHX15) complex to the subject.

As used herein, an “inhibitor” is a molecule that binds to a substrate and decreases its activity. Such a substrate may be an enzyme, protein or small molecule. Blocking a substrate's activity can kill a pathogen or correct a metabolic imbalance. The binding of an inhibitor can stop another molecule (biomolecule) from entering the substrate's active site and/or hinder the substrate from catalyzing its reaction. Inhibitor binding is either reversible or irreversible. Irreversible inhibitors usually react with the substrate and change it chemically (e.g. via covalent bond formation). These inhibitors modify key amino acid residues needed for enzymatic activity. In contrast, reversible inhibitors bind non-covalently and different types of inhibition are produced depending on the binding and complexation. For example, the inhibition may be competitive, uncompetitive, non-competitive or mixed.

As used herein, “inhibit” refers to an act of decreasing a substrate's activity as described above. This action may be performed by a molecule which may be an inhibitor.

In one embodiment, the inhibitor of LOC-DHX15 complex is a DHX inhibitor. The DHX inhibitor may, for example, be YK-4-279. The DHX inhibitor may be a DHX15 inhibitor. Alternatively, the inhibitor of LOC-DHX15 complex may be an LOC inhibitor.

The inhibitor as referred to herein includes and encompasses any active agent that reduces the accumulation, function or stability of DHX; or decrease expression of DHX gene. The inhibitor may also include any active agent that reduces the accumulation, function or stability of LOC RNA; or decrease expression of LOC gene. The inhibitor may also include any active agent that directly disrupt LOC-DHX15 interaction. Such inhibitors include without limitation, small molecules and macromolecules such as nucleic acids, peptides, polypeptides, peptidomimetics, carbohydrates, polysaccharides, lipopolysaccharides, lipids or other organic (carbon containing) or inorganic molecules.

In some embodiments, the DHX inhibitor is an antagonistic nucleic acid molecule that functions to inhibit the transcription or translation of DHX transcripts. Representative transcripts of this type include nucleotide sequences corresponding to any one the following sequences: (1) human DHX nucleotide sequences as set forth for example in GenBank Accession Nos. NM_001358.3; (2) nucleotide sequences that share at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with any one of the sequences referred to in (1); (3) nucleotide sequences that hybridize under at least low, medium or high stringency conditions to the sequences referred to in (1); (4) nucleotide sequences that encode any one of the following amino acid sequences: human DHX amino acid sequences as set forth for example in GenPept Accession Nos. 043143.2; (5) nucleotide sequences that encode an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence similarity with any one of the sequences referred to in (4); and nucleotide sequences that encode an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with any one of the sequences referred to in (4).

Illustrative antagonist nucleic acid molecules include antisense molecules, aptamers, ribozymes and triplex forming molecules, RNAi and external guide sequences. The nucleic acid molecules can act as effectors, inhibitors, modulators, and stimulators of a specific activity possessed by a target molecule, or the functional nucleic acid molecules can possess a de novo activity independent of any other molecules.

Antagonist nucleic acid molecules can interact with any macromolecule, such as DNA, RNA, polypeptides, or carbohydrate chains. Thus, antagonist nucleic acid molecules can interact with DHX mRNA or the genomic DNA of DHX or they can interact with a DHX polypeptide. Often antagonist nucleic acid molecules are designed to interact with other nucleic acids based on sequence homology between the target molecule and the antagonist nucleic acid molecule. In other situations, the specific recognition between the antagonist nucleic acid molecule and the target molecule is not based on sequence homology between the antagonist nucleic acid molecule and the target molecule, but rather is based on the formation of tertiary structure that allows specific recognition to take place.

In some embodiments, anti-sense RNA or DNA molecules are used to directly block the translation of DHX by binding to targeted mRNA and preventing protein translation. Antisense molecules are designed to interact with a target nucleic acid molecule through either canonical or non-canonical base pairing. The interaction of the antisense molecule and the target molecule may be designed to promote the destruction of the target molecule through, for example, RNAseH mediated RNA-DNA hybrid degradation. Alternatively the antisense molecule may be designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Non-limiting methods include in vitro selection experiments and DNA modification studies using DMS and DEPC. In specific examples, the antisense molecules bind the target molecule with a dissociation constant (K_{d}) less than or equal to 10⁻⁶, 10⁻⁸, 10⁻¹⁰, or 10⁻¹². In specific embodiments, antisense oligodeoxyribonucleotides derived from the translation initiation site, e.g., between −10 and +10 regions are employed.

Aptamers are molecules that interact with a target molecule, suitably in a specific way. Aptamers are generally small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with Kds from the target molecule of less than 10⁻¹² M. Suitably, the aptamers bind the target molecule with a Kd less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is desirable that an aptamer have a K_{d} with the target molecule at least 10-, 100-, 1000-, 10,000-, or 100,000-fold lower than the K_{d} with a background-binding molecule. A suitable method for generating an aptamer to a target of interest (e.g., PHD, FIH-1 or vHL) is the “Systematic Evolution of Ligands by EXponential Enrichment” (SELEX™). The SELEX™ method is described in U.S. Pat. Nos. 5,475,096 and 5,270,163 (see also WO 91/19813). Briefly, a mixture of nucleic acids is contacted with the target molecule under conditions favorable for binding. The unbound nucleic acids are partitioned from the bound nucleic acids, and the nucleic acid-target complexes are dissociated. Then the dissociated nucleic acids are amplified to yield a ligand-enriched mixture of nucleic acids, which is subjected to repeated cycles of binding, partitioning, dissociating and amplifying as desired to yield highly specific high affinity nucleic acid ligands to the target molecule.

In other embodiments, anti-DHX ribozymes are used for catalyzing the specific cleavage of DHX RNA. The mechanism of ribozyme action involves sequence specific hybridization of the ribozyme molecule to complementary target RNA, followed by a endonucleolytic cleavage. There are several different types of ribozymes that catalyze nuclease or nucleic acid polymerase type reactions, which are based on ribozymes found in natural systems, such as hammerhead ribozymes, hairpin ribozymes, and tetrahymena ribozymes. There are also a number of ribozymes that are not found in natural systems, but which have been engineered to catalyze specific reactions de novo. Representative ribozymes cleave RNA or DNA substrates. In some embodiments, ribozymes that cleave RNA substrates are employed. Specific ribozyme cleavage sites within potential RNA targets are initially identified by scanning the target molecule for ribozyme cleavage sites, which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base pairing. Triplex molecules are preferred because they can bind target regions with high affinity and specificity. It is generally desirable that the triplex forming molecules bind the target molecule with a K_{d} less than 10⁻⁶, 10⁻⁸, 10⁻¹⁰, or 10⁻¹².

External guide sequences (EGSs) are molecules that bind a target nucleic acid molecule forming a complex, and this complex is recognized by RNAse P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA: EGS complex to mimic the natural tRNA substrate. Similarly, eukaryotic EGS/RNAse P-directed cleavage of RNA can be utilized to cleave desired targets within eukaryotic cells.

In other embodiments, RNA molecules that mediate RNA interference (RNAi) of a DHX gene or DHX transcript can be used to reduce or abrogate gene expression. RNAi refers to interference with or destruction of the product of a target gene by introducing a single-stranded or usually a double-stranded RNA (dsRNA) that is homologous to the transcript of a target gene. RNAi methods, including double-stranded RNA interference (dsRNAi) or small interfering RNA (siRNA), have been extensively documented in a number of organisms, including mammalian cells and the nematode C. elegans (Fire et al., 1998. Nature 391, 806-811). In mammalian cells, RNAi can be triggered by 21- to 23-nucleotide (nt) duplexes of small interfering RNA (siRNA) (Chiu et al., 2002 Mol. Cell. 10:549-561; Elbashir et al., 2001. Nature 411:494-498), or by micro-RNAs (miRNA), functional small-hairpin RNA (shRNA), or other dsRNAs which are expressed in vivo using DNA templates with RNA polymerase III promoters (Zeng et al., 2002. Mol. Cell 9:1327-1333; Paddison et al., 2002. Genes Dev. 16:948-958; Lee et al., 2002. Nature Biotechnol. 20:500-505; Paul et al., 2002. Nature Biotechnol. 20:505-508; Tuschl, T., 2002. Nature Biotechnol. 20:440-448; Yu et al., 2002. Proc. Natl. Acad. Sci. USA 99 (9): 6047-6052; McManus et al., 2002. RNA 8:842-850; Sui et al., 2002. Proc. Natl. Acad. Sci. USA 99 (6): 5515-5520).

In specific embodiments, dsRNA per se and especially dsRNA-producing constructs corresponding to at least a portion of a DHX gene are used to reduce or abrogate its expression. RNAi-mediated inhibition of gene expression may be accomplished using any of the techniques reported in the art, for instance by transfecting a nucleic acid construct encoding a stem-loop or hairpin RNA structure into the genome of the target cell, or by expressing a transfected nucleic acid construct having homology for a DHX gene from between convergent promoters, or as a head to head or tail to tail duplication from behind a single promoter. Any similar construct may be used so long as it produces a single RNA having the ability to fold back on itself and produce a dsRNA, or so long as it produces two separate RNA transcripts, which then anneal to form a dsRNA having homology to a target gene.

Absolute homology is not required for RNAi, with a lower threshold being described at about 85% homology for a dsRNA of about 200 base pairs (Plasterk and Ketting, 2000, Current Opinion in Genetics and Dev. 10:562-67). Therefore, depending on the length of the dsRNA, the RNAi-encoding nucleic acids can vary in the level of homology they contain toward the target gene transcript, i.e., with dsRNAs of 100 to 200 base pairs having at least about 85% homology with the target gene, and longer dsRNAs, i.e., 300 to 100 base pairs, having at least about 75% homology to the target gene. RNA-encoding constructs that express a single RNA transcript designed to anneal to a separately expressed RNA, or single constructs expressing separate transcripts from convergent promoters, are suitably at least about 100 nucleotides in length. RNA-encoding constructs that express a single RNA designed to form a dsRNA via internal folding are usually at least about 200 nucleotides in length.

The promoter used to express the dsRNA-forming construct may be any type of promoter if the resulting dsRNA is specific for a gene product in the cell lineage targeted for destruction. Alternatively, the promoter may be lineage specific in that it is only expressed in cells of a particular development lineage. This might be advantageous where some overlap in homology is observed with a gene that is expressed in a non-targeted cell lineage. The promoter may also be inducible by externally controlled factors, or by intracellular environmental factors.

In some embodiments, RNA molecules of about 21 to about 23 nucleotides, which direct cleavage of specific mRNA to which they correspond, as for example described by Tuschl et al. in U.S. 2002/0086356, can be utilized for mediating RNAi. Such 21- to 23-nt RNA molecules can comprise a 3′ hydroxyl group, can be single-stranded or double stranded (as two 21- to 23-nt RNAs) wherein the dsRNA molecules can be blunt ended or comprise overhanging ends (e.g., 5′, 3′).

In some embodiments, the antagonist nucleic acid molecule is a siRNA. siRNAs can be prepared by any suitable method. For example, reference may be made to International Publication WO 02/44321, which discloses siRNAs capable of sequence-specific degradation of target mRNAs when base-paired with 3′ overhanging ends, which is incorporated by reference herein. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER™ siRNA Construction Kit.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. In addition, methods for formulation and delivery of siRNAs to a subject are also well known in the art. See, e.g., US 2005/0282188; US 2005/0239731; US 2005/0234232; US 2005/0176018; US 2005/0059817; US 2005/0020525; US 2004/0192626; US 2003/0073640; US 2002/0150936; US 2002/0142980; and US2002/0120129, each of which is incorporated herein by reference.

The present invention also contemplates small molecule agents that binds to or reduce the RNA helicase activity of DHX. Candidate agents encompass numerous chemical classes, though typically they are organic molecules, preferably small organic compounds having a molecular weight of more than 50 and less than about 2,500 Dalton. Candidate agents comprise functional groups necessary for structural interaction with proteins, particularly hydrogen bonding, and typically include at least an amine, carbonyl, hydroxyl or carboxyl group, desirably at least two of the functional chemical groups. The candidate agent often comprises cyclical carbon or heterocyclic structures or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogues or combinations thereof.

Small (non-peptide) molecule modulators of DHX are particularly advantageous. In this regard, small molecules are desirable because such molecules are more readily absorbed after oral administration, have fewer potential antigenic determinants, or are more likely to cross the cell membrane than larger, protein-based pharmaceuticals. Small organic molecules may also have the ability to gain entry into an appropriate cell and affect the expression of a gene (e.g., by interacting with the regulatory region or transcription factors involved in gene expression); or affect the activity of a gene by inhibiting or enhancing the binding of accessory molecules.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogues.

Screening may also be directed to known pharmacologically active compounds and chemical analogues thereof. Screening for DHX inhibitors according to the invention can be achieved by any suitable method. For example, the method may include contacting a cell expressing a polynucleotide corresponding to a gene that encodes a DHX with an agent suspected of having the modulatory activity and screening for the modulation of the level or functional activity of the DHX, or the modulation of the level of a transcript encoded by the polynucleotide, or the modulation of the activity or expression of a downstream cellular target of the polypeptide or of the transcript (hereafter referred to as target molecules). Detecting such modulation can be achieved utilizing techniques including, but not restricted to, ELISA, cell-based ELISA, inhibition ELISA, Western blots, immunoprecipitation, slot or dot blot assays, immunostaining, RIA, scintillation proximity assays, fluorescent immunoassays using antigen-binding molecule conjugates or antigen conjugates of fluorescent substances such as fluorescein or rhodamine, Ouchterlony double diffusion analysis, immunoassays employing an avidin-biotin or a streptavidin-biotin detection system, and nucleic acid detection assays including reverse transcriptase polymerase chain reaction (RT-PCR).

It will be understood that a polynucleotide from which a DHX is regulated or expressed may be naturally occurring in the cell which is the subject of testing or it may have been introduced into the host cell for the purpose of testing. In addition, the naturally-occurring or introduced polynucleotide may be constitutively expressed-thereby providing a model useful in screening for agents which down-regulate expression of an encoded product of the sequence wherein the down regulation can be at the nucleic acid or expression product level. Further, to the extent that a polynucleotide is introduced into a cell, that polynucleotide may comprise the entire coding sequence that codes for the a DHX or it may comprise a portion of that coding sequence (e.g., the active site of the DHX) or a portion that regulates expression of the corresponding gene that encodes the DHX (e.g., a DHX promoter). For example, the promoter that is naturally associated with the polynucleotide may be introduced into the cell that is the subject of testing. In this instance, where only the promoter is utilized, detecting modulation of the promoter activity can be achieved, for example, by operably linking the promoter to a suitable reporter polynucleotide including, but not restricted to, green fluorescent protein (GFP), luciferase, β-galactosidase and catecholamine acetyl transferase (CAT). Modulation of expression may be determined by measuring the activity associated with the reporter polynucleotide.

These methods provide a mechanism for performing high throughput screening of putative modulatory agents such as proteinaceous or non-proteinaceous agents comprising synthetic, combinatorial, chemical and natural libraries. These methods will also facilitate the detection of agents which bind either the polynucleotide encoding the target molecule or which modulate the expression of an upstream molecule, which subsequently modulates the expression of the polynucleotide encoding the target molecule. Accordingly, these methods provide a mechanism of detecting agents that either directly or indirectly modulate the expression or activity of a target molecule according to the invention.

Compounds may be further tested in the animal models to identify those compounds having the most potent in vivo effects. These molecules may serve as “lead compounds” for the further development of pharmaceuticals by, for example, subjecting the compounds to sequential modifications, molecular modeling, and other routine procedures employed in rational drug design.

The methods as defined herein may further comprise administering a chemotherapy to the subject. The chemotherapy may be a standard-of-care chemotherapy. For example, the chemotherapy may be temozolomide (TMZ). The chemotherapy may be an anti-cancer agent as described herein.

In one embodiment, the method comprises inhibiting a glioma stem cell in the subject. The glioma stem cell may be a stem cell expressing LOC105375914 RNA. The glioma stem cell may be a cell expressing elevated levels of LOC105375914 RNA.

In one embodiment, there is provided an inhibitor of the LOC-DHX15 complex in the manufacture of a medicament for treating a glioma in a subject.

In one embodiment, there is provided the use of an inhibitor of LOC-DHX15 complex in the manufacture of a medicament for the treatment of glioma in a subject.

In certain embodiments, the present invention provides compositions, for example pharmaceutical compositions. The term “pharmaceutical composition,” as used herein, refers to a composition comprising at least one active agent as described herein, and one or more other components useful in formulating a composition for delivery to a subject, such as diluents, buffers, saline (such as phosphate buffered saline), cell culture media, carriers, stabilizers, dispersing agents, suspending agents, thickening agents, excipients, preservatives, and the like. “Pharmaceutical compositions” permit the biological activity of the active agent, and do not contain components that are unacceptably toxic to the living subject to which the composition would be administered.

Provided herein is a pharmaceutical composition comprising an inhibitor of the LOC-DEAH-box helicase 15 (DHX15) complex. The pharmaceutical composition may further comprise TMZ. The pharmaceutical composition may comprise a pharmaceutically acceptable carrier. By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Representative pharmaceutically acceptable carriers include any and all solvents, dispersion media, coatings, surfactants, antioxidants, preservatives {e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

Pharmaceutical compositions can be in numerous dosage forms, for example, tablet, capsule, liquid, solution, soft-gel, suspension, emulsion, syrup, elixir, tincture, film, powder, hydrogel, ointment, paste, cream, lotion, gel, mousse, foam, lacquer, spray, aerosol, inhaler, nebulizer, ophthalmic drops, patch, suppository, and/or enema. The choice of dosage forms and excipients will depends upon the active agent to be delivered and the specific disease or disorder to be treated or prevented, and can be selected by one of ordinary skill in the art without having to engage in any undue experimentation.

Also provided herein are compositions for performing one or more methods as defined herein. The compositions may include probes, amplification oligonucleotides, and the like.

Also provided herein are kits for performing one or more methods as defined herein. The kits may include probes, amplification oligonucleotides, and the like. The kits may further comprise one or more buffers and reagents, together with instructions for use.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

Certain embodiments of the invention will now be described with reference to the following examples which are intended for the purpose of illustration only and are not intended to limit the scope of the generality hereinbefore described.

### EXAMPLES

### Methods

### Cell Culture and Treatment

GL261-Luc cells were cultured in RPMI medium and all other cell lines were maintained in Dulbecco's Modified Eagle's Medium (DMEM) supplemented with 10% FBS (Gibco) and penicillin/streptomycin (Gibco) and grown at 37° C. with 5% CO2 using standard cell culture techniques. Cells were treated either 10 ng/ml TNFα (Calbiochem) or 100 ng/ml LPS (Sigma, 595 L2654) for the indicated time points.

### RNA-Seq Data Analysis

Raw RNA expression data (RSEM matrix) of 1018 gliomas patients' samples and 20 non-gliomas samples were downloaded from Chinese Glioma Genome Atlas (CGGA). IDH mutation status of 966 samples were obtained. Differential genes expression analysis was performed by using glmFit( ) function in edgeR V.3.28.1 package. Significant differentially expressed genes (DEGs) were defined as genes with expression fold change ≥2 and a false discovery rate (FDR)<0.05. With reference to KEGG database, over-represented pathways were measured separately for up- and down regulated genes using ClusterProfiler V.3.14.3 package.

scRNA-Seq Data Analysis

Raw count matrixes of single cell RNA-Seq (scRNA-Seq) data from 9 GBM patients were downloaded from NCBI GEO (GSE117891). With reference to expression data of the same patients from bulk RNA-Seq, the scRNA-Seq samples were separated into three groups based on the expression levels of LOC. Downstream data analysis below were done using functions in Seurat v3.2.3 R package. Expression normalization and scaling were first implemented before performing dimensional reduction analysis using RunPCA( ) and RunTSNE( ) functions. All cells were then clustered based on the expression profile. Gene markers representing each cluster were identified and by comparing to database of known cell type markers (CellMarkerdatabase), the cell type of each clusters are classified. Proportion of each cell type were then calculated using R and Student's t-test was used to test the significance of proportion shift between patients with high and low LOC expression.

RNA Interference and Real-Time qPCR

For siRNA knock-down, control siRNA and siRNA specific to LOC (CAACCTCTCTAATCAGTCTCTTTCT (SEQ ID NO: 1)) were purchased from IDT (Integrated DNA Technologies) as dicer-substrate siRNA. siRNA was transfected into T98G cells in Opti-MEM using Lipofectamine RNAiMAX siRNA transfection reagent according to manufacturer instructions and the medium was replenished with fresh DMEM after 8 hours. After 72 hours cells were harvested and total RNA was extracted by Trizol and column purified using RNeasy Mini Kit for gene expression analysis. 1 μg RNA was used as a template for reverse transcription reaction using Maxima first strand cDNA synthesis kit (Thermo Scientific). Then real-time PCR was performed from diluted cDNAs using SsoAdvanced Universal SYBR Green Supermix (BioRad). Relative gene expression was analyzed by AACT method by normalizing experimental Ct values to β-actin. For lentivirus mediated knockdown, lines were transduced with pLKO-puro lentiviral vectors containing shRNAs against LOC transcripts (LOC shRNA #1: GGAATAATGATAGCAACTACT (SEQ ID NO: 2) and LOC shRNA #2: GTTGCTAACAGTATGAGTTCT (SEQ ID NO: 3), in the presence of 5 μg/ml polybrene and subsequently selected for 72 hrs with puromycin.

### 5′ and 3′ RACE:

5′ and 3′ Rapid Amplification of cDNA Ends (RACE) for loc 105375914 was performed. Briefly for 5′, to generate cDNA templates reverse transcription was performed from 1 μg of total RNA of A375 cells using SuperScript II Reverse Transcriptase using gene specific primer for reverse transcription (acgatgccaagtegtctttt (SEQ ID NO: 4). For appending a Poly-A tail to 5′ site of cDNA products, terminal transferase reaction was performed with Terminal deoxynucleotidyltransferase. This 5′ end tailed cDNA pool was used for nested PCR amplification using Q5 High Fidelity DNA polymerase (NEB). Second PCR product was cloned into pLenti CMV GFP Puro vector using Xbal and SalI sites. At least 10 different independent bacterial clones were Sanger sequenced to obtain 5′ end site of LOC and sequences were blasted to human genome for mapping. For 3′ RACE, to generate cDNA pool the same protocol as described above was used with minor changes. For reverse transcription 100 ng/μl of QT-Primer (ccagtgagcagagtgacgaggactcgagctcaagcttttttttttttttttt (SEQ ID NO: 5)) was used and RNA-primer mix containing reverse transcription components first incubated at 25° C. for 5 minutes and incubate in the same as discussed above. This cDNA pool was diluted to 1 ml with RNase/DNase free water and used for first set of amplification using same protocol for 5′ RACE. Second PCR product was cloned into Puc19 vector suing Xbal and SalI sites. At least 10 different independent bacterial clones was sanger sequenced to obtain 3′ end site of loc105375914 and sequences were blasted to human genome for mapping.

### Removal of LOC Promoter Region by CRISPR/Cas9 Editing

pX458-GFP plasmid was modified by removing Cas9-GFP and inserting DsRed (pX458-DsRed) gene sequence under Cbh promoter to be able to select double-positive cells in FACS.gRNA1 was cloned into pX458-GFP and gRNA2 was cloned into px458-DsRed plasmids. Cells were co-transfected in 6-well plate using X-tremeGENE 9 transfection reagent (Sigma). Double positive single cells were sorted into 96-well plate (1cell/well) by MoFlo XDP 4 Laser system (Beckman Coulter) and each clone was genotyped by PCR using outward primers from targeting region. For Gm16685 deletion genotyping primers as follows F: GCATTCCCTTAGGTAGACCTCC (SEQ ID NO: 6) and Reverse: GGGAGTGATTATGGGTGGTGAG (SEQ ID NO: 7) and for LOC deletion genotyping Forward: ATTAAGCTCCGGGAGGACAT (SEQ ID NO: 8) and Reverse: CAGGGTCCTGGGAGTGACTA (SEQ ID NO: 9). The presence of deletion for each positive clone was also validated by Sanger sequencing.

### Western Blot Analysis

Total protein was extracted using Totex buffer (20 mM Hepes at pH 7.9, 0.35M NaCl, 20% glycerol, 1% NP-40, 1 mM MgCl2, 0.5 mM EDTA, 0.1 mM EGTA, 50 mM NaF, and 0.3 mM NaVO3) supplemented with complete protease and phosphatase inhibitor cocktail (Roche). Immunoblotting was performed with following antibodies: anti-p-p38 (Thr180/Tyr182) 3D7 (Cell signalling; #9215S), anti-p38 (Santa Cruz; #sc-728), anti-p-p65 (Ser536) (Cell signalling; #3031L), anti-p65 (Santa Cruz; #sc-8008), anti-actin (Sigma; #A2066), anti-HSP90α/β (F-8) (Santa Cruz; #sc-13119), anti-PPM1D (Santa Cruz; #sc-376257), anti-PPM1D (Santa Cruz; #sc-376257), p-IKKα/β (Ser176/180) (Cell signalling; #2697S), IKKα/β (H-470) (Santa Cruz; #7607), (Origene; #sc-TA190113).

### GBM Patient-Derived Specimens and Primary Cell Culture

After receiving informed consent, tumor specimens or malignant ascites with corresponding clinical records were obtained from patients undergoing surgery or paracentesis at Samsung Medical Center (SMC) in accordance with its Institutional Review Board (IRB file #201004004). Patient-derived primary GBM cells were cultured as previously described. For sphere culture, GSCs were cultured in the “NBE” neurosphere culture condition.

### Limiting Dilution Assays (LDA).

For LDA, cells were plated with control lentivirus or knock down lentivirus. Infected cells and control cells were plated in 96-well plates. After 2˜3 weeks, the number of wells without spheres were counted. At the time of quantification, each well was examined for the formation of tumor spheres. Stem cell frequency was calculated using extreme limiting dilution analysis (ELDA) (http://bioinf.wehi.edu.au/software/elda/).

### PDC-Based Chemical Screening and Analysis

Tumorsphere forming PDCs, cultured in serum-free medium, were dissociated into single cells and seeded into 384-well plates (500 cells/well). PDCs were treated with TMZ in 2 mM. After 6 days of incubation at 37° C. in a 5% CO2 humidified incubator, cell viability was accessed using adenosine triphosphate (ATP) monitoring system based on firefly luciferase (ATPLite™ 1step, PerkinElmer) and estimated by EnVision Multilabel Reader (PerkinElmer). Relative cell viability for each dose was obtained by normalization with dimethyl sulfoxide (DMSO).

### Glioma Orthotopic Models

All mouse experiments were performed according to the guidelines of the Animal Use and Care Committees at the Samsung Medical Center and Association for Assessment and Accreditation of Laboratory Animal Care-accredited guidelines. 6 weeks old female BALB/c nude mice were used for intracranial transplantation. Patient-derived glioma cells (1×105 per mouse) were injected into the brains of mice by stereotactic intracranial injection (coordinates: 2 mm anterior, 2 mm lateral, 2.5 mm depth from the dura). Mice were sacrificed either when 25% body weight loss or neurological symptoms (lethargy, ataxia, and seizures) were observed.

### Syngeneic Glioma Mouse Model

6 weeks old female Gm16685 WT and Gm16685 KO mice were used for intracranial transplantation. Basically, 25000 cells (GL261-Luc Gm16685 WT or GL261-Luc Gm16685 KO) in a volume of 2 μl CO₂ independent medium (Thermo fisher. #18045088) into the striatum; 2 mm left of the sagittal suture and 0.5 mm anterior to the bregma at a depth of 3 mm from the dura, using a 2.5 μl Hamilton syringe equipped with an unbeveled 33G needle. Mice were sacrificed either when 25% body weight loss or neurological symptoms (lethargy, ataxia, and seizures) were observed.

### Fluorescence In Situ Hybridization

Quasar 570-conjugated Stellaris oligonucleotide probes against LOC were obtained from LGC Biosearch Technologies (Petaluma, CA). Cells were hybridized with the Stellaris RNA FISH probe sets following the manufacturer's instructions. Briefly, cells were fixed for 10 min at room temperature with 4% formaldehyde solution in PBS. After fixation, cells were placed in 70% (vol./vol.) ethanol for 4 h at 4° C. Aspirate the 70% ethanol and wash buffer was added for 5 min. The probe was diluted at a concentration of 125 nM in hybridization buffer. Hybridization solution with probes was added to each sample and then placed at 37° C. overnight. The samples were then washed with wash buffer twice for 5 min each at 37° C. DAPI was added before mounting and imaging.

### In Vitro RNA-Protein Interaction Followed by Mass Spectrometry

The RNA protein interaction assay was performed as previously described. Briefly LOC sense and antisense, and human telomerase RNA (Terc) was in vitro transcribed using biotin RNA labeling mix (Roche) and T7 RNA polymerase (Promega). Biotin labeled

RNA probes were folded by adding equal volumes RNA structure buffer (20 mM Tris [pH 7.0], 0.2M KCl, and 20 mM MgCl2), heated at 70° C. for 5 minutes and cooled down at room temperature for 30 minutes to allow secondary structure formation. Cells were treated with TNFα for 1.5 hours and sonicated in RIP buffer (150 mM KCl, 25 mM Tris pH: 7.4, 0.5 mM DTT, 0.5% NP-40, 1 mM PMSF, Promega recombinant RNasin ribonuclease inhibitor (150 unit per 1 ml), 50 mM NaF, 0.3 mM NaVO3, and complete protease inhibitor). Subsequently, cell lysate was pre-cleared with streptavidin agarose beads (Invitrogen) for 1 hour at 4° C. Pre-cleared protein lysate were incubated with either 3 μg folded LOC probe or Terc probe for 4 hours at 4° C. with rotation and 2 additional hours with the streptavidin-agarose beads. Next beads were washed for 5 times with RIP buffer and proteins were retrieved by boiling beads in 40 μl of 2× NuPAGE LDS Sample Buffer for 10 minutes. The supernatant was collected into a new microfuge tube after centrifugation at 1000 rpm for 3 minutes at room temperature. These eluted samples (30 μl) were analyzed by mass spectrometry and 10 μl of remaining eluted sample was processed for silver staining using ProteoSilver Silver Stain Kit (Sigma).

### Sample Processing and Mass Spectrometry Analysis

Samples were run on a 4-12% NuPage Novex Bis-Tris Gel (Invitrogen). Gels were subsequently stained with Colloidal Blue Staining Kit (Invitrogen). Protein bands were excised and after protein extraction trypsin digestion was carried out. Samples were analyzed on an Orbitrap (Thermo Fisher) with parameters as follows: Survey full scan MS spectra ranging m/z 310-1400 were acquired. This was acquired at a resolution of r=60,000 at m/z 400, an AGC target of 1e6, and a maximum injection time of 500 ms. Top ten intense peptide ions were selected and sequentially fragmented in the linear ion trap by collision induced dissociation with a normalized collision energy of 35%. A dynamic exclusion was applied using a maximum exclusion list of 500 with one repeat count and exclusion duration of 30 s. Data was searched using X! Tandem Vengeance (2015.12.15.2) with the following: fixed modification on cysteine carbamidomethyl, variable modifications on oxidized methionine and N-acetylation and, maximum missed cleavages of 2, parent ion tolerance of 10 ppm and fragment ion tolerance of 0.5 Da-searched against the human and human decoy database (185868 entries). Spectrum counts of peptides and proteins were derived using Scaffold Proteomics Software (version 3, Matrix Science) with 95% confidence interval and minimum of 2 peptides as criteria.

### MS2 Pull-Down Assay

LOC-MS2 vector or Terc-MS2 vector co-transfected with MS2-GFP plasmids into 293T cells. 48 hours later cells were harvested and lysed in IP lysis buffer (50 mM Tris-HCl pH 8.0, 150 mM NaCl, 1% NP-40, 0.5% sodium deoxycholate, 0.1% SDS). Cell lysates were incubated with GFP antibody for 6 hours and then immuno-precipitated with Protein G Sepharose beads (GE Healthcare) overnight. The beads were washed three times with washing buffer (10 mM Tris-HCl pH 7.5.1 mM EDTA, 1 mM EGTA, 150 mM NaCl, 1% Triton X-100). Immunoprecipitated proteins were eluted by boiling the beads in 2×LDS buffer (Invitrogen). Immunoblotting was performed as described above with following antibodies: anti-GFP antibody (1:1000, Invitrogen; #A-11122), anti-DHX15 antibody (1:1000, Santa Cruz; #sc-271686).

### RNA Immunoprecipitation

T98G cells were washed with ice-cold PBS in 6-well plate and lysed in 100 μl of RIP lysis buffer (50 mM Tris pH: 8, 150 mM NaCl, 0.5% NP-40, 0.5% Sodium deoxycholate, 0.05% SDS, supplemented with protease inhibitor cocktail and 100 U/ml RNase Inhibitor). Cell lysates were collected into microfuge tubes and further incubated on ice for 20 minutes. Subsequently, cells were sonicated with Bioruptor for 5 minutes and centrifuged for 15 min at maximum speed. Next supernatants were transferred into a clean tube and were immunoprecipitated overnight with DHX15 antibody at 4° C. The following day, Dynabeads™ Protein G was added and incubated for 3 hours. Beads were washed three times with IP wash buffer (10 mM Tris pH: 7.5, 1 mM EDTA, 1 mM EGTA, 150 mM NaCl, 1% Trition-X). RNA was extracted and isolated from the beads using TRIzol reagent. RNA was reverse transcribed and enrichment of RNAs was quantified by RT-qPCR using % input method.

Cross-Linking Immunoprecipitation and qPCR

Cells were UV-cross-linked according to previously published protocols (71) (81) (83) (87). Briefly, 293T overexpressing DHX15 cells were irradiated at 150 mJ/cm2 in a CL-1000 UVP UVcross-linker and then subjected to cell lysis buffer (50 mM Tris-HCl pH 7.4,100 mM NaCl,1% NP-40, 0.1% SDS and 0.5% sodium deoxycholate) in the presence of protease and RNase inhibitors. DNA was removed from the cell lysate by Turbo DNase treatment and RNA was fragmented by 5 min-treatment with RNase I at 37° C. The cleared lysates were incubated with flag M2 beads overnight at 4° C., and beads were washed in lysis buffer. Proteinase K was added and incubated at 55° C. for 30 minutes. Total RNA was isolated using the Qiagen RNA Mini Kit with DNase I treatment. After RNA isolation, RT-qPCR was performed using 12 primer pairs covering the full-length LOC or 4 pairs for Terc. Data is normalized to control vector.

### Immunoprecipitation Assay

Cells were harvested and lysed in IP lysis buffer (50 mM Tris-HCl pH 8.0, 150 mM NaCl, 1% NP-40, 0.5% sodium deoxycholate, 0.1% SDS). Protein concentration was measured by Bradford method. DHX15, p65 or p38 was immunoprecipitated after incubating cell lysate with antibody for 6 hours and an additional 2 hours with Protein G Sepharose beads (GE Healthcare). The beads were washed three times with washing buffer (10 mM Tris-HCl pH 7.5, 1 mM EDTA, 1 mM EGTA, 150 mM NaCl, 1% Triton X-100) and immunoprecipitated proteins were eluted by boiling the beads in 2×LDS buffer (Invitrogen) for 10 minutes. Immunoblotting was performed as described above with the following antibodies: anti-PPM1D antibody (1:1000, Santa Cruz; #sc-376257), anti-p65 antibody (1:1000, Santa Cruz; sc-8008) and anti p38 antibody (1:1000, Santa Cruz; sc-728).

### Cell Viability Assay

For the cell viability assay, 5000 cells per well were plated in 96-well plates and treated with serially diluted DHX inhibitor (0 to 200 μM) or TMZ (0 μM to 1600 μM). Cell viability was measured using the Cell Counting Kit-8 (Dojindo). The IC50 values were calculated as the mean drug concentration required to inhibit cell proliferation by 50% compared with vehicle treated controls.

### Drug Treatment in Glioma Xenograft Model

In brief, GBM cells LN18 were first engineered to express a luciferase protein according to previous protocol. For testing DHX inhibitor in wtIDH1 and mIDH1 GBM cells, a total number of 2.5×105 wtIDH1 LN18-Luc or mIDH1 LN18-Luc cells in 5 μl PBS were intracranially injected into brains of 6-week-old female NSG mice (Invivos). 6 mice were injected for each group. Mice with established orthotopic xenografts were randomized to treatment with vehicle (10% DMSO, 40% PEG400, and 50% PBS) or 20 mg/kg DHX inhibitor once daily. For combination treatment, after tumors were established at day 8 to 10, mice were randomized to treatment with vehicle or 20 mg/kg DHX inhibitor once daily or TMZ once daily (20 mg/kg) via intraperitoneal injection starting from day 8 for 5 days or both. Tumor growth was assessed using an IVIS Spectrum imager (PerkinElmer), and survival dates until the onset of neurologic symptoms were recorded for survival curves.

### Animal Studies

All animal studies were conducted in accordance with the Institutional Animal Care and Use Committee at A*STAR (Singapore). All procedures were approved under the IACUC protocol ID #221680 and ID #201572.

### Statistical Analysis

Two tailed Student's t-test was perform for statistical analysis of between two groups. Two way ANOVA was used for statistical analysis of tumor volume. PRISM software version 7 was used to plot graphs and for statistical analysis.

### Example 1

### Results

TNF/NFκB Singalling Serve as a Central Signalling Hub in wtIDH1 Glioma

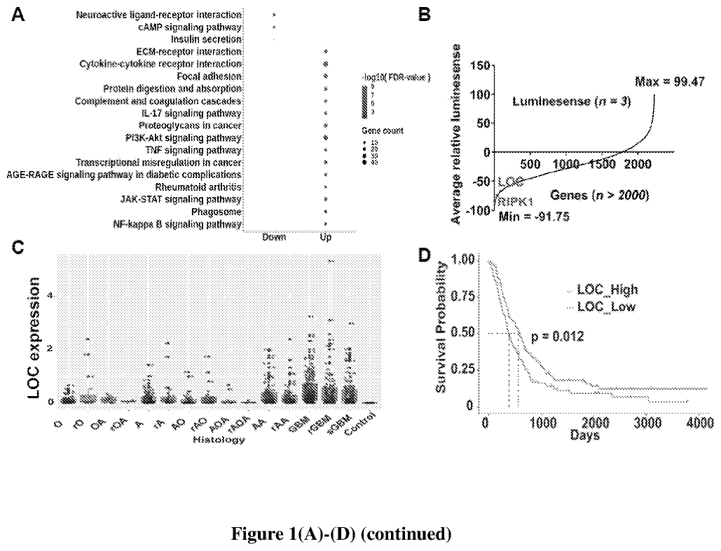

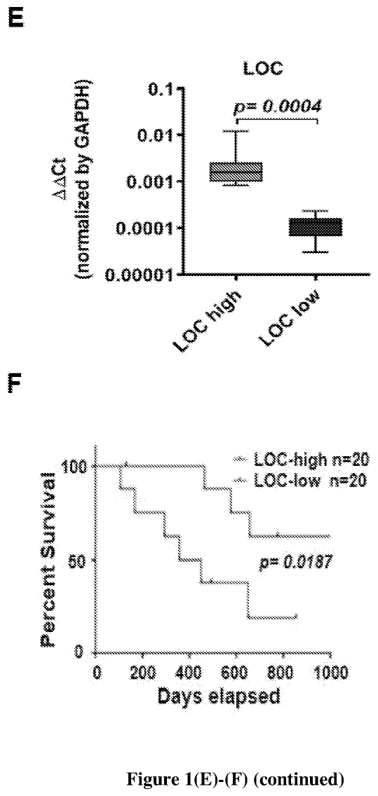

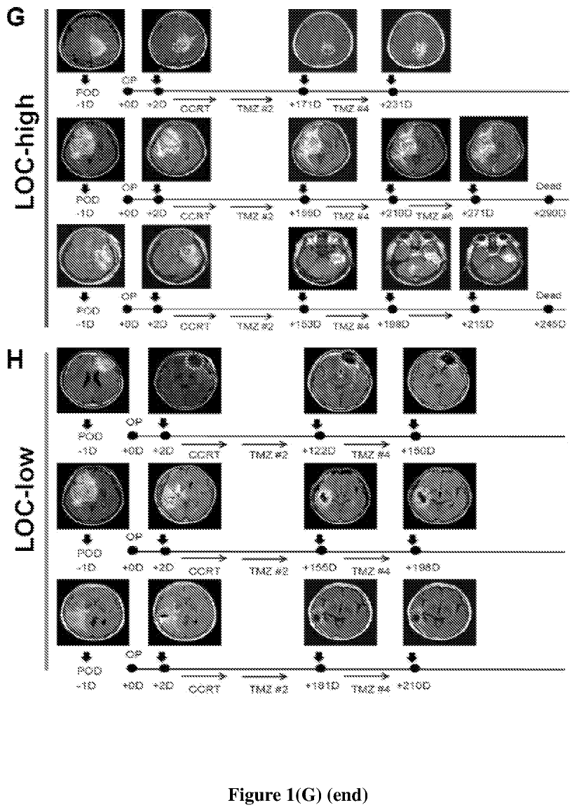

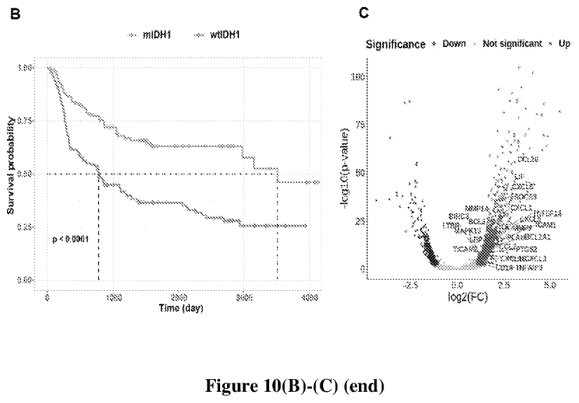

Mutations in IDH1 are prevalent in human malignancies, such as glioma. The landscape of somatic mutations from the cohort also revealed that mIDH1 are hotspot mutation which accounts for about 47% of the patients. Interestingly, the absence of IDH1 mutations predicts an unfavourable disease outcome with worse median survival (FIG. 10B). To systemically characterize targetable vulnerabilities in wtIDH1 glioma, transcriptome analyses using RNA-seq data from wtIDH1 and mIDH1 glioma patients were conducted. KEGG pathway analysis recovered that TNF/NFκB signalling was highly activated in mIDH1 glioma, suggesting that TNF/NFκB signalling could act as a potential dependency in wtIDH1 glioma (FIG. 1A).

### High Throughput Screening of Novel NFκB Regulators

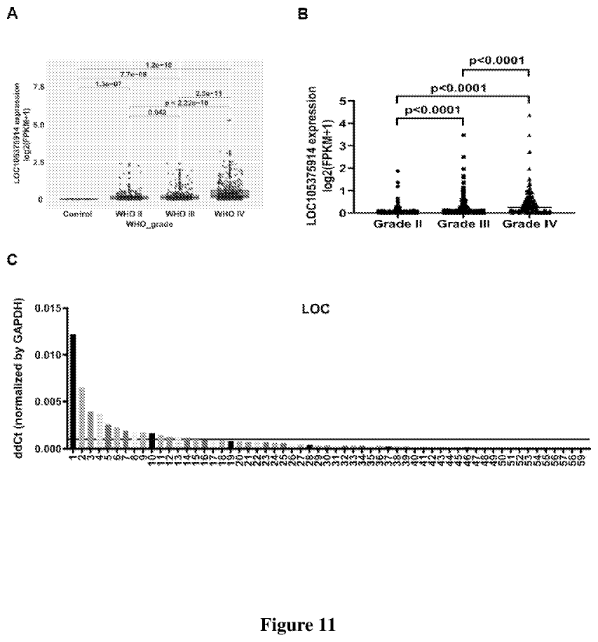

As TNF/NFκB singalling is involved in many house-keeping function, the study aimed to identify novel NFκB regulators which could work in context specific manner. To do so, high throughput screening of lncRNAs using NFκB luciferase reporter cell lines has been performed. Results of the primary screening targeting over 2000 lncRNAs are shown (FIG. 1B). A well-known NFκB regulator RIPK1 acts as a positive control. It was found that LOC (LOC105375914), a novel lncRNA located in the anti-sense direction of IL-7 gene, could execute an indispensable role in glioma by fine-tuning NFκB activity. Using 5′ and 3′ RACE and sequencing, LOC, as a transcript of 1508 nucleotides (SEQ ID NO: 10) with 4 exons, located on human chromosome 8q21.13 (+) was identified. LOC is highly expressed in high grade glioma especially in GBM (FIG. 1C, FIG. 11A, B). To assess the clinical significance of LOC in GBM, the expression of LOC in 219 GBM tumours was analyzed based on RNA-seq data and the patients were grouped based on LOC expression levels as LOC-high (n=108) or LOC-low (n=111) (FIG. 1D). Patients with high LOC expression had significantly lower survival rate compared to low LOC expression (FIG. 1D). To further evaluate the clinical significance of LOC in another independent cohort, the expression of LOC in 59 GBM patient tumors was analyzed by RT-qPCR and the patients were grouped based on LOC expression levels as LOC-high (n=20) or LOC-low (n=20) (FIG. 1E, FIG. 11C). Similarly, patients with high LOC expression displayed a significantly lower survival rate compared to ones with low LOC expression (FIG. 1F). Furthermore, Magnetic Resonance Imaging (MRI) data after surgical resection and standard therapy showed that the resected tumor always came back, and reduced the survival significantly in patients with high LOC expression (FIG. 1G, resection marked by dotted lines). However, patients with low LOC expression had a better clinical prognosis after surgical resection and standard therapy, due to reduced tumor growth post-therapy (FIG. 1H, marked by dotted lines). These findings indicate that LOC expression correlated with poor prognosis in GBM patients.

### LOC Overexpression Confers Drug Resistance and Promotes Tumorigenesis in GBM Patient-Derived Cells

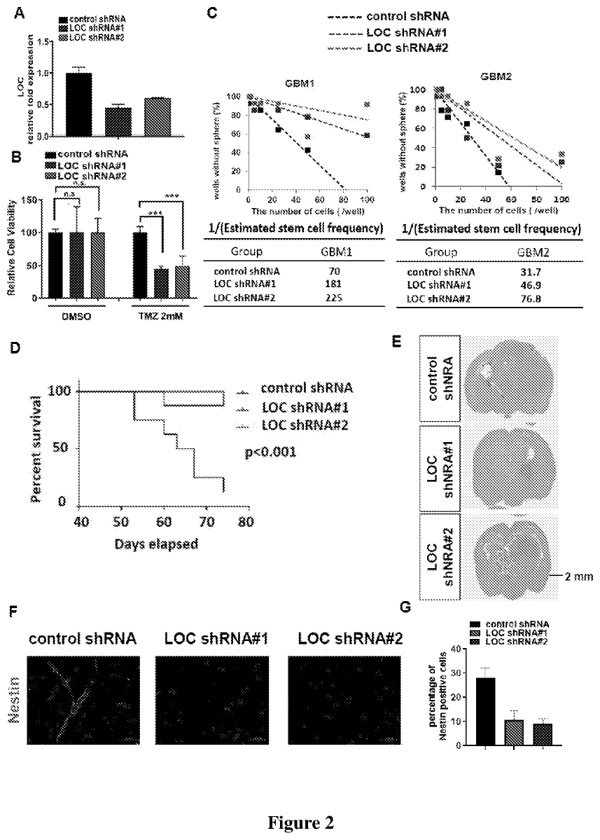

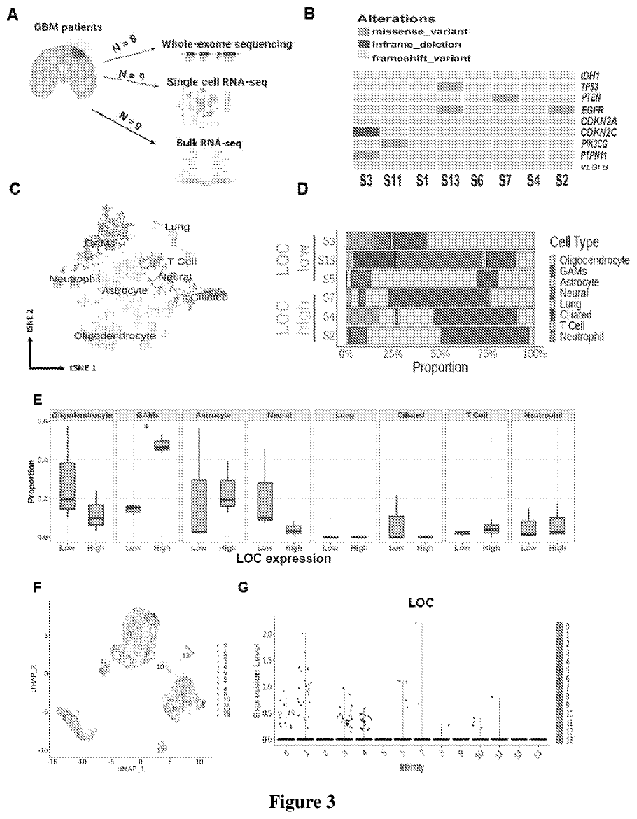

The role of LOC in cancer development and drug resistance of human glioma was further investigated. TMZ is commonly used for the treatment of GBM patients in the clinic. However, about 50% of patients develop resistance over the course of treatment. To evaluate the role of LOC on chemo-resistance, a stable LOC knockdown GBM patient-derived primary cells were generated using two individual shRNAs. Those cells were treated with or without TMZ. Cells infected with LOC shRNAs showed a dramatic decrease in cell viability in response to TMZ but not the control group (FIG. 2A, B), suggesting that LOC could contribute to TMZ resistance. Recently, cancer stem cells have been shown to contribute to chemotherapy resistance. The stemness of GBM cells with and without LOC was analyzed by in vitro tumorsphere formation with limiting dilution assay (LDA) using two different sets of GBM patient-derived primary cells. LDA clonogenic significance showed that LOC depletion impaired stemness in 2 independent GBM patient-derived cells (FIG. 3C). In line with in vitro experiments, depletion of LOC leads to declined tumorigenicity in patient derived xenograft (PDX) models in vivo (FIG. 3D, E). Staining of cancer stem cell marker Nestin in tumors from PDX models further indicates the dampened tumor formation in the LOC-deficient group (FIG. 3F, G). These results highlighted the potential oncogenic role of LOC mediating cell proliferation, stemness, and drug resistance in GBM.

High LOC Expression Correlates with Greater Infiltration of GAMs in TME

To further decipher the role of LOC in the real tumor microenvironment, the tumor ecosystem was charted by implementing scRNA-seq (FIG. 3A). Whole-exome sequencing (WES) as well as bulk RNA sequencing (RNA-seq) were conducted using matched glioblastoma patient materials (FIG. 3A). The somatic genomic landscape of glioblastoma revealed previously reported genes such as TP53, PTEN, EGFR and PIK3CA but not IDH1 (FIG. 3B), which allows the study to mainly focus on the wtIDH1 group as glioma IDH1 mutation status has been reported to shape the brain TME. Unsupervised clustering using Louvain community detection revealed 7 clusters with distinct gene expression patterns within TME (FIG. 3C). A specific lung cluster from a lung squamous cell carcinoma patient acts as a control, which highlights the specificity of cell populations derived from glioblastoma patients (FIG. 3C). Glioblastoma patients were categorized into groups of LOC-low (S3, S5, S13) and LOC-high (S2, S4, S7) based on LOC expression (FIG. 3D). Analysis of clusters revealed remarkable changes in the immune composition (FIG. 3D). In particular, pronounced alterations in the phenotype and proportions of myeloid cells were observed, including the increased presence of GAMs (FIG. 3D-E) in the LOC-high group, suggesting a potential role of LOC in myeloid infiltrate. Next, cellular sources of LOC within the tumor niche using scRNA-seq data were explored with a higher resolution (FIG. 3F-G). Profiling of LOC expression revealed that LOC is mainly expressed in glioma cells and immune cells, particularly in GAMs (FIG. 3G), suggesting LOC could play a versatile role in both glioma cells and immune cells.

Deletion of Mouse Orthologue of LOC from Both Tumor and Host Compartments Leads to the Most Profound Frequency of Tumor Regressions

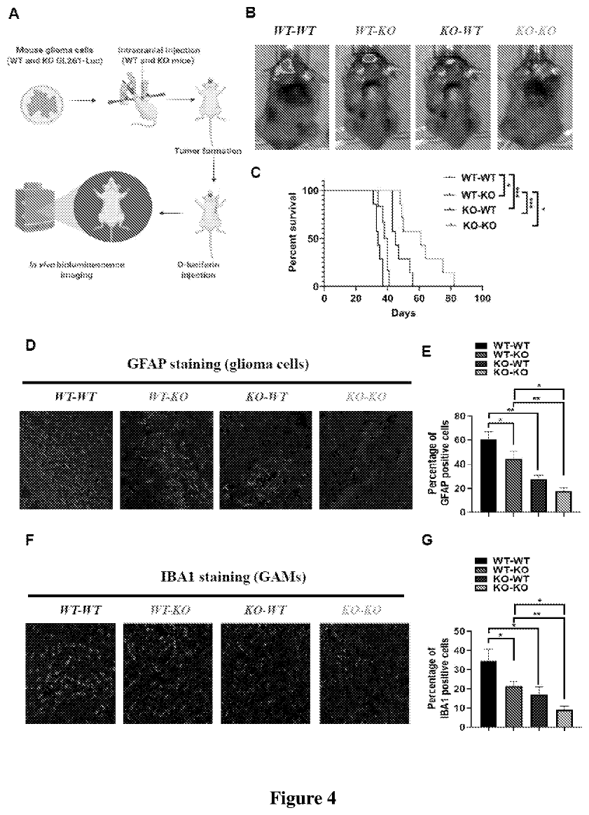

Having observed the potential applications of LOC in reprogramming the TME, these findings were next corroborated in the set of mouse glioma TME using a syngeneic mouse model (FIG. 4A). An evolutionarily conserved lncRNA, namely Gm16685, which serves as a mouse orthologue of LOC was previously discovered. The same strategy was applied to generate isogenic mouse glioma cell line GL261-Luc by deleting p65 binding motifs (the same method of generating Gm16685 knockout mice). The deletion of the p65 motif was confirmed by genotyping PCR. To test whether both tumor and host cells derived LOC/Gm16685 could play critical roles in TME, the inventors intracranially injected WT (WT-WT, WT-KO) or KO (KO-WT, KO-KO) GL261-Luc cells into WT and KO mice and compared tumor growth in GL261 models with Gm16685 deficiency on the tumor, the host, and both compartments based on luminescence intensity (FIG. 4B). In line with a previous observation, Gm16685 loss in the tumor or host compartment led to tumor regressions and prolonged survival (FIG. 4B-C). However, the highest rate of tumor regressions and the most favorable survival were observed when neither the tumor nor the host cells expressed Gm16685 (FIG. 4B-C), indicating the indispensable role of Gm16685/LOC in the glioblastoma tumor-immune microenvironment. Immunofluorescence staining of glioma cells (GFAP) and GAMs (IBA1) further highlighted the synergistic tumor-promoting effect of Gm16685/LOC on both tumor and host compartments (FIG. 4D-G).

Human GBMs with Higher LOC Expression Display Enhanced NFκB Signature

Next, the underlying mechanism of how LOC contributes to GBM progression was explored. As LOC has been identified as a novel regulator of NFκB from the screening, the correlation between LOC expression and NFκB gene signature in GBM was evaluated. The available in-house RNA-seq data from the LOC-high (n=12) and LOC-low (n=12) group of patients was analyzed. Overall, NFκB target genes were highly expressed in GBM patients who had higher expression of LOC compared to GBM patients with low LOC expression levels, and this is correlated with a significant negative impact on overall survival (FIG. 1F). To evaluate the functional relevance of LOC, loss and gain of function studies in patient-derived primary cancer cells was evaluated. Knockdown of LOC by shRNAs in GBM patient-derived primary cells repressed the expression of downstream target genes of NFκB signaling pathways, namely TNFα and IL-8 (not shown). In gliomas, IL8 is known to be a crucial mediator responsible for NFκB induced tumor progression. Subsequently, over expressing LOC in LOC deficient primary cancer cells rescued TNFα and IL-8 expression (FIG. 4E-G). Given that NFκB signaling is a well-known contributor of stemness, cell survival and resistance to chemotherapy, these results together show that the LOC-NFκB axis is an underlying reason for the poorer clinical outcome of patients with high LOC expression.

### LOC Regulates Phosphorylation of p65 and p38

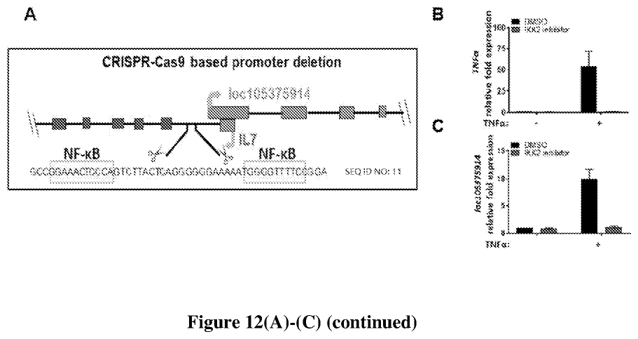

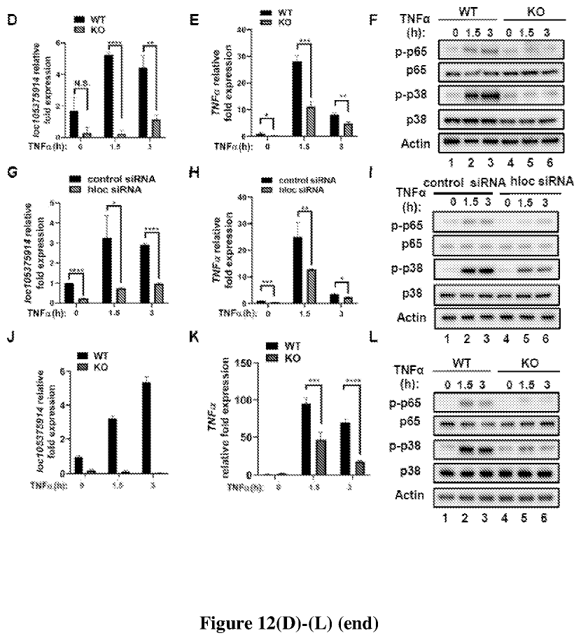

The detailed mechanistic basis of LOC action was further investigated. First of all, two NFκB binding sites in the promoter region of LOC (also located in the first intron of IL-7) were found, which suggests LOC might be regulated NFκB (FIG. 12A). Subsequent experiment using NFκB inhibitor revealed that LOC can be induced by TNFα and this induction can be impeded by blocking NFκB activity (FIG. 12B, C), indicating the potential positive feedback loop between LOC and NFκB. To further elucidate the detailed mechanism of this feedback loop, isogenic cell lines were generated by removing NFκB motifs using CRISPR-Cas9 genome editing. LOC expression was abolished in LN18 KO cells (FIG. 12D), and LN18 KO cells also showed a dramatic reduction of NFκB target gene such as TNFα (FIG. 12E). Interestingly, upon treatment with TNFα, decreased phosphorylation of p65 and p38 (FIG. 12F) was observed but not other upstream events (data not shown) in KO cells. These results suggest that LOC might be regulating NFκB activity through p65 and p38 activation, and hence modulating expression of NFκB and p38 target genes such as TNFα. To exclude the off-target effect by CRISPR genome editing, knock-down experiments by siRNA in GBM cancer cell lines LN18 (FIG. 12G-I) were employed. In accordance with results obtained from primary GBM cells and human GBM cell lines, blunting LOC expression dampened NFκB target genes such as TNFα (FIG. 12H) and NFκB/p38 activation (FIG. 12I), This observation was extended to other cell types and it was found that LOC expression was no longer induced in response to TNFα in T98G-KO cells (FIG. 12.J). Indeed, compared to WT cells, TNFα expression in LOC KO cells was dramatically lower (12K). It was found p65 and p38 activation were also reduced in LOC KO cells compared to WT cells (FIG. 12L), suggesting the generality of the observations. Furthermore, ectopic expression of LOC was sufficient to rescue the loss of p65 and p38 phosphorylation after TNFα treatment in LOC KO cells (not shown). These results further confirmed that the observed differences in LOC deficient cells are mediated by LOC and not due to changes in genomic structure or alterations in neighbouring genes caused by genome editing.

Identification of DHX15 RNA Helicase as a Binding Partner of LOC lncRNA

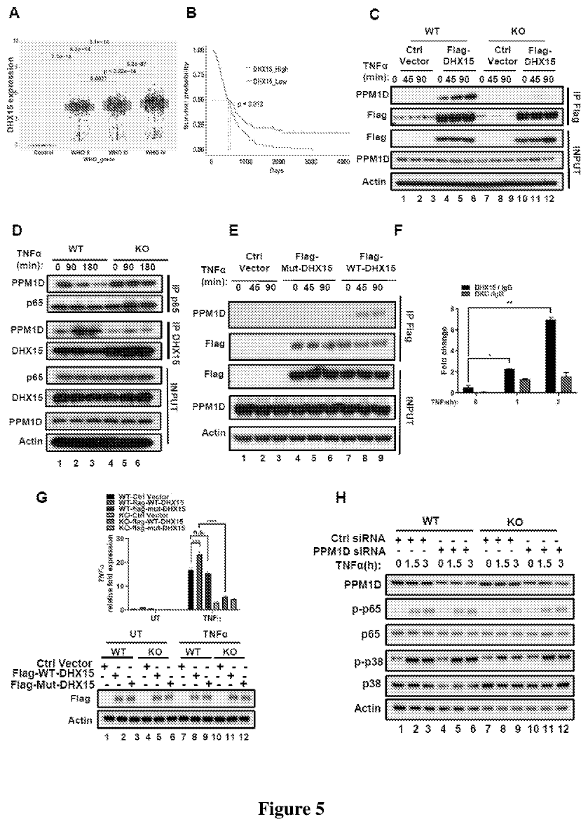

FISH analysis of LOC and Gm16685 revealed that both of them were mainly localized in the cytoplasm. It was attempted to identify LOC interacting proteins using RNA pull-down assays by incubating in vitro transcribed biotinylated LOC with protein extracts of TNFα treated cells. Biotinylated RNA protein complexes were pulled down using streptavidin-agarose beads and resolved on SDS-PAGE gel for visualization by silver staining, or sent for mass-spectrometry. In vitro transcribed and biotinylated human telomerase RNA (Terc) was used as a positive control. As expected, RNA pull-down of Terc specifically brought dyskerin (DKC) protein, compared to bead control, reassuring the robustness of the experimental conditions. To prioritize candidate interactors after mass-spectrometry, the dataset was filtered such that there is zero or no exclusive unique spectrum count in bead control and at least three exclusive unique spectrum counts in LOC probe with TNFα treated conditions. Analysis of LOC interactome by mass-spectrometry identified DHX15 (DEAH box RNA helicase family member), a pre-mRNA-splicing factor ATP-dependent RNA helicase, as a potential interacting partner. Indeed, the analysis revealed that DHX15 is highly expressed in high-grade gliomas including GBM (FIG. 5A) and patients with high DHX15 expression display significantly lower survival compared to patients with low DHX15 expression (FIG. 5B). These data suggest that DHX15 may play a vital role in GBM by complexing LOC.

LOC Licenses Active DHX15 for Sequestering PPM1D Phosphatase Away from its Substrates p65

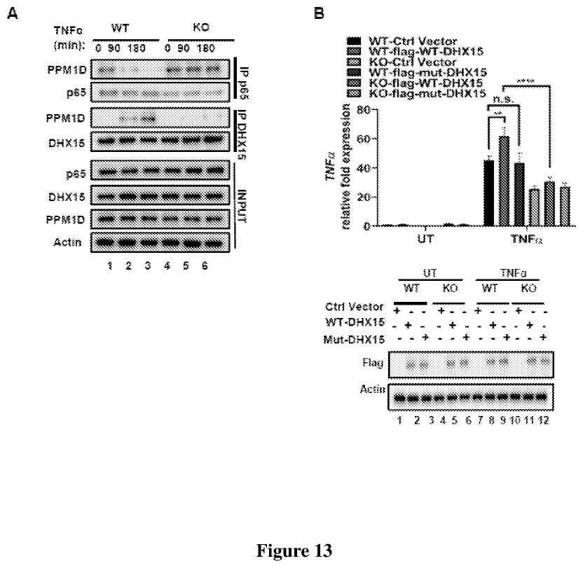

Previous studies have been shown that PPMID phosphatase negatively regulates inflammatory transcriptional programs by de-phosphorylating p65 or p38 kinase activation. Indeed, decreased phosphorylation of p65 and p38 was seen in the absence of LOC (results not shown). It was proposed that PPMID might bind LOC and de-phosphorylate p65 and p38. However, PPMID was not shown to be acted as an RNA binding protein in previous literature. It was then hypothesized that DHX15 might be involved in PPM1D mediated p65 and p38 de-phosphorylation. To test the hypothesis that DHX15 could bind PPM1D, DHX15 was immunoprecipitated and it was found that DHX15 interacts with PPMID and this interaction was augmented upon TNFα treatment in WT cells (FIG. 5C, lane 4-6). However, this interaction was significantly disrupted in LOC KO cells (FIG. 5C, lane 10-12). Furthermore, immunoprecipitation of DHX15 in LOC KO cells ectopically expressing full length or 3′ truncated LOC (43′-loc) showed that 3′ region of LOC is essential for the DHX15-PPMID interaction (results not shown). Those results collectively suggest that LOC is essential to license the DHX15 interaction with PPM1D. Similar to pull-down results obtained from ectopically expressed DHX15, immunoprecipitation of endogenous DHX15 pulled down PPM1D in WT cells but significantly less in LOC KO cells (FIG. 5D, FIG. 13A). Interestingly, it was observed that the helicase activity of DHX15 is required for optimal PPM1D and DHX15 interaction since TNFα induced DHX15-PPMID interaction was impaired when a helicase dead mutant version of DHX15 was used in the pull-down assay (FIG. 5E). TNFα treatment also enhanced the interaction of LOC with DHX15 (FIG. 5F), mirroring the interaction of PPMID with DHX15 (FIG. 5C, lanes 4-6), suggesting that LOC: DHX15 induced by TNFα is required for squelching PPM1D and that levels of LOC are limiting in forming this complex in unstimulated cells (FIG. 5C, lanes 1-3) and the levels of this complex increase after TNFα stimulation (FIG. 5A, lanes 4-6). The requirement for LOC and DHX15 helicase activity for the interaction of DHX15 with PPM1D suggests that LOC provides an RNA scaffold for DHX15 binding of PPM1D, but this interaction is only successful when the LOC RNA is opened up by DHX15 helicase activity. Indeed, if that is the case, it is hypothesised that the DHX15-LOC complex is responsible for sequestering PPM1D away from p65 and p38 in TNFα induced cells. To test this, PPM1D-p65 interaction was analyzed in TNFα treated WT cells. PPM1D interacted avidly with p65 in untreated cells, presumably to keep p65 inactive, but this interaction was reduced upon TNFα treatment (FIG. 5D, lanes 1-3, FIG. 13A, lanes 1-3), possibly due to concomitant LOC expression and formation of a LOC DHX15 scaffold (FIG. 5F). However, in LOC KO cells, PPMID remained firmly bound to p65 (FIG. 5D, lanes 4-6, FIG. 13A, lanes 1-3), suggesting that in the absence of LOC, DHX15 cannot sequester PPM1D away from p65 due to the lack of the LOC-DHX15 scaffold. This could be the basis of reduced p-p65 and p-p38 levels seen in LOC KO cells throughout this study. Indeed, both DHX15 and PPMID are barely associated in unstimulated cells, and it is the activation and binding of LOC, levels of which are limiting, that licenses DHX15 to sequester PPM1D upon NFκB activation.

To address whether LOC lncRNA acts in trans to mediate these effects, it was ectopically expressed in LOC KO cells. Reconstitution of LOC in LOC KO cells rescued the phosphorylation of p38 and p65, targets of PPM1D (not shown). These results also further confirm that LOC acts in trans to regulate PPM1D targets via DHX15. To further explore if DHX15 executes its action through LOC, wt-DHX15 and mut-DHX15 were expressed in WT and LOC KO cells. Indeed, activation of NFκB targets such as TNFα was observed when wt-DHX15 not mut-DHX15 was expressed in WT cells (FIG. 5G). However, activation of NFκB targets was significantly dampened when DHX15 was expressed in LOC KO cells (FIG. 5G). These results indicate that LOC lncRNA can exert its function in trans by acting as a scaffold for complexing with DHX15 to sequester PPM1D phosphatase away from its substrates. To further understand if PPM1D mediates its action mainly via LOC, it was also shown that PPM1D knockdown restored the phosphorylation of p38 and p65 in LOC KO cells, further suggesting that PPM1D is a crucial determinant of LOC action (FIG. 5H). It is possible that other substrates of PPMID may also play a role in the described phenomenon consequent to the induction of LOC. Given that this induction is dependent on the p65 subunit of NFκB, LOC could be the critical licensing factor required to turn on full-blown inflammatory responses in an evolutionarily conserved fashion.

wtIDH1 Glioma Cells are More Susceptible to Pharmacological Inhibition of LOC: DHX15-PPMID-NFκB Axis.

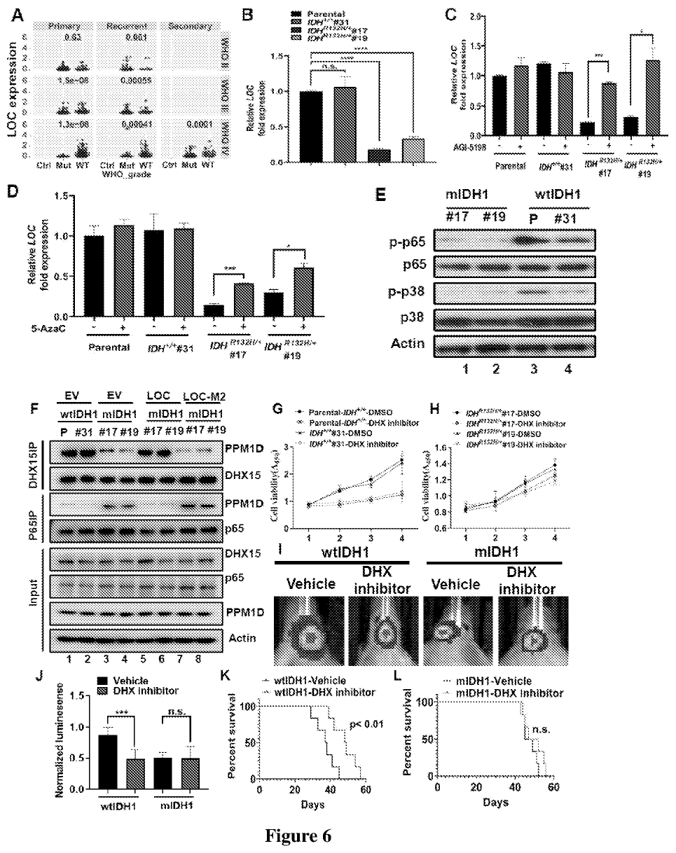

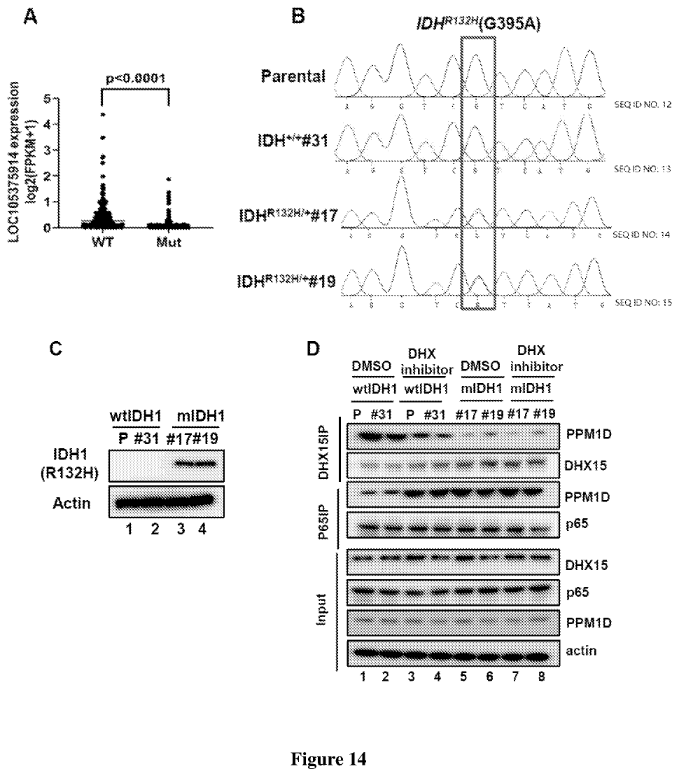

Next, it was surveyed whether LOC: DHX15 axis could account for NFκB-dependent transcriptional addition in wtIDH1 glioma cells. Profiling of LOC expression in wtIDH1 and mIDH1 glioma patients across different WHO grade revealed that LOC was highly expressed in wtIDH1 high-grade gliomas (FIG. 6A, FIG. 14A). To further evaluate the effect of IDH1 mutation on LOC expression, single base editing was employed to introduce heterozygous IDH1 R132H mutation (IDH1^{R132H/WT}) with a single base substitution of guanine (G) to adenosine (A) in human GBM cells. Heterozygous IDH1 R132H point mutation was validated by Sanger sequencing (FIG. 14B) and western blot analysis with a specific antibody against R132H IDH1 (FIG. 14C). LOC expression was significantly blunted by IDH1 R132H mutation in two independent clones (FIG. 6B). In addition, the administration of selective mutant IDH1^{R132H} inhibitor restored the LOC expression in IDH1^{R132H/WT} cells (FIG. 6C). As IDH1 mutation is known to induce a DNA hypermethylation phenotype, it was explored whether LOC dysregulation could be triggered by this epigenetic reprogramming. Similarly, 5-AzaC (5-Azacytidine, a DNA methyltransferase inhibitor to inhibit DNA methylation) treatment enabled to abrogate this hypermethylation phenotype, suggesting that LOC expression could be diminished by IDH1 mutation induced hypermethylation phenotype (FIG. 6D). It was hypothesized that LOC could be a key factor for the activation of TNF/NFκB signalling in wtIDH1 gliomas. A significant elevation of phosphorylation of p65 and p38 was also observed, indicating enhanced activity of TNF/NFκB in wtIDH1 gliomas (FIG. 6E). Furthermore, declined p65-PPMID interaction and pronounced DHX15-PPM1D interaction was detected in wtIDH1 GBM cells compared to mIDH1 GBM cells, suggesting LOC: DHX15 axis is crucial for switching on TNF/NFκB signalling in wtIDH1 gliomas cells (FIG. 6F, lane 3-4 vs. land 1-2). Ectopic expression of wild-type LOC but not the mutant version of LOC (mutant LOC cannot bind to DHX15) in mIDH1 cells was able to complex with DHX15 to sequester PPM1D from its substrate p65 (FIG. 6F, lane 5-6 vs. land 3-4 and FIG. 6F, lane 7-8 vs. land 3-4). Administration of DHX inhibitor could specifically reverse this phenotype in wtIDH1 GBM cells by dampening DHX15-PPM1D interaction (FIG. 14C, lane 3-4 vs. land 1-2) and enhancing p65-PPMID complexing (FIG. 14C, lane 3-4 vs. land 1-2) but not in mIDH1 GBM cells (FIG. 14C, lane 7-8 vs. land 5-6). Consistently, DHX inhibitor treatment can significantly inhibit cell growth in the wtIDH1 group (FIG. 6G) but not in the mIDH1 group (FIG. 6H). Additionally, lessened tumor growth (FIG. 6I, J) and prolonged survival (FIG. 6K, L) were only seen in the wtIDH1 group but not in the mIDH1 group further underlines that RNA: RNA Helicase LOC: DHX15 could serve as a promising vulnerability in wtIDH1 gliomas cells (FIG. 6H-I).

LOC: DHX15-PPMID-NFκB Axis Confers TMZ Resistance in wtIDH1 Glioma Cells

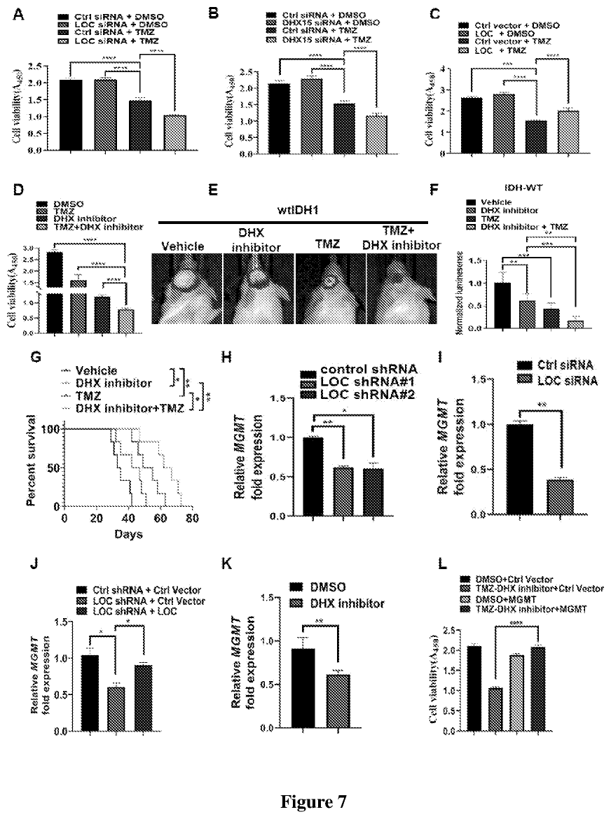

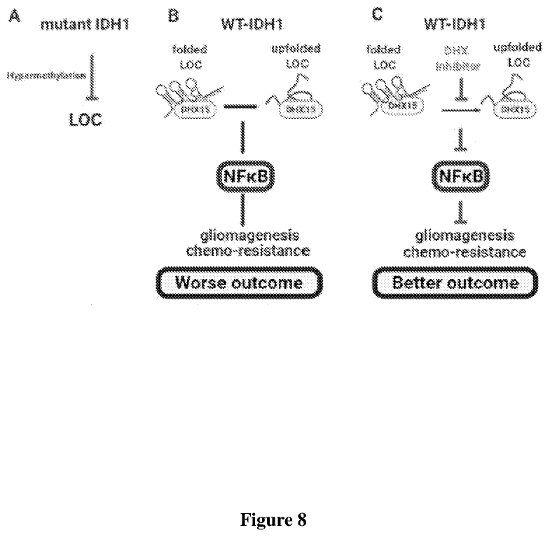

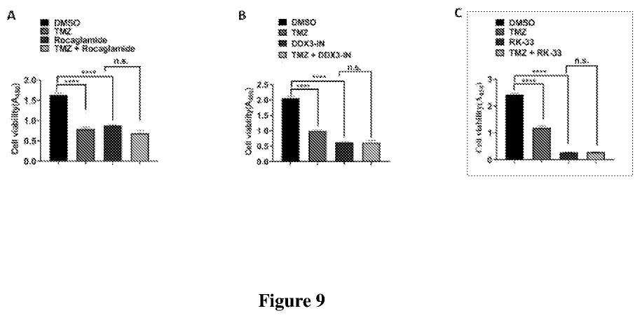

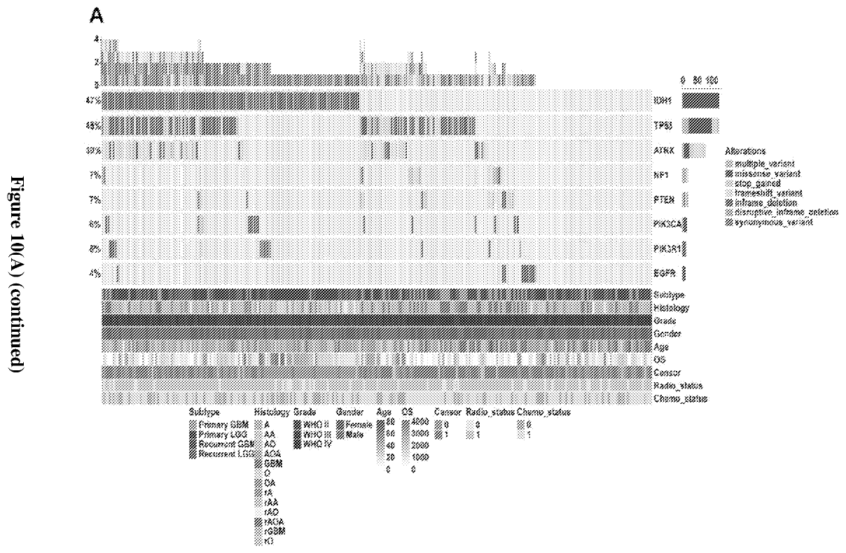

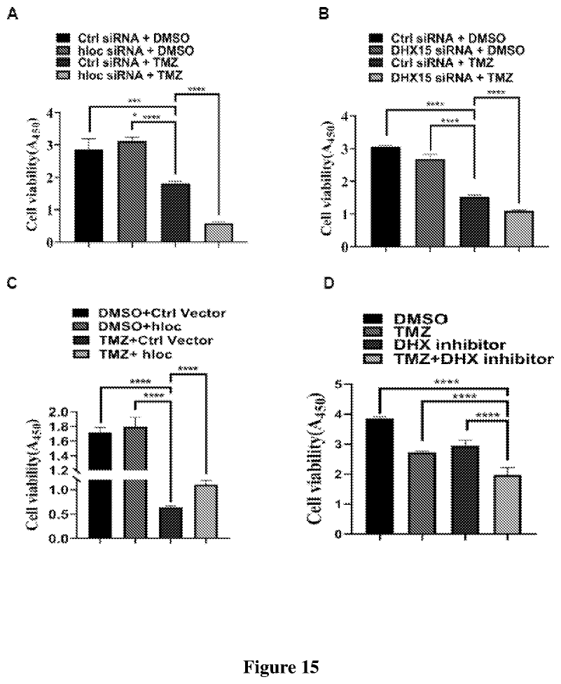

MRI data and drug response data emphasized the essential role of LOC in conferring TMZ resistance. To further investigate if LOC: DHX15-PPMID-NFκB axis plays a role in TMZ resistance, loss of and gain of function studies were employed in wtIDH1 GBM cells. As a result, depletion of LOC (FIG. 7A, FIG. 15A) or DHX15 (FIG. 7B, FIG. 15B) by siRNAs sensitizes GBM cells to TMZ. Ectopic expression of LOC leads to higher resistance to TMZ (FIG. 7C, FIG. 15C). Given the potential effect of LOC: DHX15-PPM1D-NFκB axis in TMZ response, it was investigated whether TMZ combined with DHX inhibitor could have a synergistic effect (FIG. 7D, FIG. 15D). The results indicate that combination treatment produced a more pronounced effect rather than a single drug treatment in two different GBM cells (FIG. 7D, FIG. 15D). This combination therapeutic strategy was applied into in vivo xenograft model. In consistent with in vitro experiments, augmented inhibition of tumor growth in TMZ combined with DHX inhibitor group (FIG. 7E, F) and improved survival rate (FIG. 7G) was also observed. MGMT (O-6-Methylguanine-DNA Methyltransferase) has been described to be the well-known factor leading to resistance of TMZ due to its direct role in counteracting DNA alkylation damage in glioma. A significant reduction of MGMT expression upon LOC knockdown using shRNAs or siRNAs (FIG. 7H, I) was observed and this reduction can be further abrogated by overexpression of LOC (FIG. 7J). Similarly, MGMT expression also can be blunted by the administration of DHX inhibitor (FIG. 7K). Most importantly, the inhibition of cell growth upon TMZ combination with DHX inhibitor treatment was abrogated by MGMT overexpression, demonstrating MGMT is indeed crucial for LOC: DHX15-PPMID-NFκB axis induced TMZ resistance (FIG. 7L). These results collectively suggest that LOC: DHX15-PPMID-NFκB axis exerts its critical role in TMZ resistance and could serve as a promising druggable target. The study shows that combinational therapy of TMZ and DHX inhibitor is an effective treatment for GBM patients.

1. A method of determining the prognosis of a glioma in a subject, the method comprising detecting LOC105375914 RNA in a glioma sample obtained from the subject, wherein an elevated level of LOC105375914 RNA as compared to a reference indicates that the subject has a high grade glioma and/or is likely to have a poor prognosis.

2. The method of claim 1, wherein the method comprises detecting wild-type isocitrate dehydrogenase 1 (IDH1).

3. The method of claim 1 or 2, wherein the high grade glioma is a World Health Organization (WHO) Grade III or IV glioma.

4. The method of any one of claims 1 to 3, wherein the high grade glioma is Glioblastoma multiforme (GBM).

5. The method of any one of claims 1 to 4, wherein the high grade glioma is likely to be resistant to chemotherapy.

6. The method of any one of claims 1 to 5, wherein the subject with the high grade glioma has a likelihood of cancer recurrence.

7. The method of any one of claims 1 to 6, wherein the reference is a sample from a healthy subject.

8. A method of identifying a high grade glioma in a subject, the method comprising: detecting LOC105375914 RNA in a cancer sample obtained from the subject, wherein an elevated level of LOC105375914 RNA as compared to a reference indicates that the subject has a high grade glioma.

9. A method of identifying and treating a high grade glioma in a subject, the method comprising: a) detecting LOC105375914 RNA in a cancer sample obtained from the subject, wherein an elevated level of LOC105375914 RNA as compared to a reference indicates that the subject has a high grade glioma; and b) administering an anti-cancer agent to the subject found to have high grade glioma to treat the high grade glioma.

10. A method of predicting a likelihood of resistance to chemotherapy in a subject suffering from a glioma, the method comprising detecting LOC105375914 RNA in a glioma sample obtained from the subject, wherein an elevated level of LOC105375914 RNA as compared to a reference indicates that the subject has a likelihood of resistance to chemotherapy.

11. The method of claim 10, wherein the chemotherapy is a standard of care therapy.

12. The method of claim 11, wherein the standard of care therapy is temozolomide (TMZ).

13. A method of predicting a likelihood of recurrence of a glioma in a subject, the method comprising detecting LOC105375914 RNA in a glioma sample obtained from the subject, wherein an elevated level of LOC105375914 RNA as compared to a reference indicates that the subject has a likelihood of recurrence.

14. The method of claim 13, wherein the subject has undergone chemotherapy, radiotherapy or surgery.

15. A method of treating a glioma in a subject by administering an inhibitor of the LOC-DEAH-box helicase 15 (DHX15) complex to the subject.

16. The method of claim 15, wherein the glioma is a high grade glioma.

17. The method of claims 15 and 16, wherein the glioma has wild-type IDH1.

18. The method of any one of claims 15 to 17, wherein the glioma expresses LOC105375914 RNA.

19. The method of any one of claims 15 to 18, wherein the inhibitor is a DHX15 inhibitor.

20. The method of any one of claims 15 to 19, wherein the method further comprises administering a chemotherapy to the subject.

21. The method of claim 20, wherein the chemotherapy comprises temozolomide.

22. The method of any one of claims 15-21, wherein the method comprises inhibiting a glioma stem cell in the subject.

23. A method of inhibiting proliferation of a glioma stem cell in a subject, the method comprising administering an inhibitor of the LOC-DEAH-box helicase 15 (DHX15) complex to the subject.


SEQUENCE LISTING (15 sequences)

SEQ ID NO: 1 - DNA, 25 bp, synthetic construct
  source 1..25: mol_type = other DNA
caacctctctaatcagtctctttct

SEQ ID NO: 2 - DNA, 21 bp, synthetic construct
  source 1..21: mol_type = other DNA
ggaataatgatagcaactact

SEQ ID NO: 3 - DNA, 21 bp, synthetic construct
  source 1..21: mol_type = other DNA
gttgctaacagtatgagttct

SEQ ID NO: 4 - DNA, 20 bp, synthetic construct
  source 1..20: mol_type = other DNA
acgatgccaagtcgtctttt

SEQ ID NO: 5 - DNA, 52 bp, synthetic construct
  source 1..52: mol_type = other DNA
ccagtgagcagagtgacgaggactcgagctcaagcttttttttttttttttt

SEQ ID NO: 6 - DNA, 22 bp, synthetic construct
  source 1..22: mol_type = other DNA
gcattcccttaggtagacctcc

SEQ ID NO: 7 - DNA, 22 bp, synthetic construct
  source 1..22: mol_type = other DNA
gggagtgattatgggtggtgag

SEQ ID NO: 8 - DNA, 20 bp, synthetic construct
  source 1..20: mol_type = other DNA
attaagctccgggaggacat

SEQ ID NO: 9 - DNA, 20 bp, synthetic construct
  source 1..20: mol_type = other DNA
cagggtcctgggagtgacta

SEQ ID NO: 10 - RNA, 1508 nt, synthetic construct
  source 1..1508: mol_type = other RNA
acttctctagggagttctgagctgcaagagcccagctactcgccgaacccgggccgcctgcgtgagtaacttggggcggccaggagcacgggaagggcgccagacagagagcctttggctgcttctgagaccggctgtgggctgccaccgctgagcggcgcggaggctgcagcgttccctctcaggacagaaagcaggcgtggggactgttactcgcagcctgccaggggcagcagccgccccaggttcaagtggctatgtgccaggatactcaggcgcggctgttggctgcccatgggagagccagtgctctccgcaggtccaaagtgcaaggccgggctattcccggacttgcctaggagcaggggcccccagcgcgtctgggattgccccggcgcgtcgggcgcgcgaacttgtgcgcaagggagaagagcgcttaccatggaacatggtctgcgggaggcgggcgtagtcatgatgaccgcaactggagcaggagcaagctctcaccgcccatagtcactcccaggaccctggtcttccgcggagttgccgagtctgtgttgggcagggtgatctctgcagctggttcctcttacccacgccgcgactgcagtttcatccatcccaaggggggcggcacacactacggcgtggctctgcgctttgccttttccataacttccgtaggatccgccagcagtgtactttcagtttctactttgcgcttggtctgcaggttcaatctttgggatcatcagcatggaatcttcattatcccctctctcctgggcacctgcttcccgcgcacctggatgaggaccagaggaattcgcgaatttccgaatcaccgcaggaaaaaccagctggcctgaatcaaagcaattctgggtaaaagacttcccagatgatctctcaacgccttggcaacgcttggcgactgggagctagaaccatgagttgttaatgtgtgcacggaagccacaagtgtgcaggacgcaagctgggttattggaagcggacgacaccaaggaggcgttgccgcggatgtggtggacgatgccaagtcgtcttttaaaaaaggaagtgaagaatgattaagagggtcacgccctctaccactccaacctctctaatcagtctctttctgctccgtctgctgtacatgcacctgctcaggtgagacctcaaggaaaaataatcctgtgggtccacctaatctgacaaccacagttgctaacagtatgagttctgcagttatacagaactaagtccattagctgtgtgacccatggccaaatattttccttctctgtgcctgcatttcttcatctgcaaactggaataatgatagcaactacttcagaggactatgctagagtgcaatggcgcaatcttggctcactgcaacctccacttcccaggttcaagtgattctcctgcctcagtctcctgagtagctgatattacaggtgcctgccactacacccagagcgagacactgcctc

SEQ ID NO: 11 - DNA, 49 bp, synthetic construct
  source 1..49: mol_type = other DNA
gccggaaactcccagtcttactcaggggggaaaaatggggttttccgga

SEQ ID NO: 12 - DNA, 11 bp, synthetic construct
  source 1..11: mol_type = other DNA
aggtcgtcatg

SEQ ID NO: 13 - DNA, 11 bp, synthetic construct
  source 1..11: mol_type = other DNA
aggtcgtcatg

SEQ ID NO: 14 - DNA, 11 bp, synthetic construct
  source 1..11: mol_type = other DNA
aggtcgtcatg

SEQ ID NO: 15 - DNA, 11 bp, synthetic construct
  source 1..11: mol_type = other DNA
aggtcgtcatg